SEQUENCE LISTING (22 sequences)

SEQ ID NO: 1 - DNA, 121 bp, Pisum sativum
  source 1..121: mol_type = genomic DNA
ctttcttctgtatttccttcttttctttttcctggccaccaaacagcaggttcatatttctcaggaaacttttcaagcataacacctaataaaggaagaggataagctttatcaagagcca

SEQ ID NO: 2 - DNA, 121 bp, Pisum sativum
  source 1..121: mol_type = genomic DNA
ctttcttctgtatttccttcttttctttttcctggccaccaaacagcaggttcatatttcgcaggaaacttttcaagcataacacctaataaaggaagaggataagctttatcaagagcca

SEQ ID NO: 3 - DNA, 121 bp, Pisum sativum
  source 1..121: mol_type = genomic DNA
tgagatcacagttactcaacatacaacttaaatgaaatataacgaattagcataaaactcaagaggagggcatacatcttcaccaattgaaacagcttcagggaagagcccgtgaatgaga

SEQ ID NO: 4 - DNA, 121 bp, Pisum sativum
  source 1..121: mol_type = genomic DNA
tgagatcacagttactcaacatacaacttaaatgaaatataacgaattagcataaaactcgagaggagggcatacatcttcaccaattgaaacagcttcagggaagagcccgtgaatgaga

SEQ ID NO: 5 - DNA, 121 bp, Pisum sativum
  source 1..121: mol_type = genomic DNA
gtttacataagattagaatgaattgatcactactatacagttttgagaaatgaaatacacaaggaatgcgttatgtacgcagaacagggaaagggaatcaagaatcggtagtggaatcgat

SEQ ID NO: 6 - DNA, 121 bp, Pisum sativum
  source 1..121: mol_type = genomic DNA
gtttacataagattagaatgaattgatcactactatacagttttgagaaatgaaatacacgaggaatgcgttatgtacgcagaacagggaaagggaatcaagaatcggtagtggaatcgat

SEQ ID NO: 7 - DNA, 121 bp, Pisum sativum
  source 1..121: mol_type = genomic DNA
ttttttggttcttctatagacatattcaactagtttgtttgcatccatggttcctgtcactgttacttttcctgtgctaaactccgtcactgcggtttgaactcctacaataatccataca

SEQ ID NO: 8 - DNA, 121 bp, Pisum sativum
  source 1..121: mol_type = genomic DNA
ttttttggttcttctatagacatattcaactagtttgtttgcatccatggttcctgtcacggttacttttcctgtgctaaactccgtcactgcggtttgaactcctacaataatccataca

SEQ ID NO: 9 - DNA, 121 bp, Pisum sativum
  source 1..121: mol_type = genomic DNA
ataccatgcaggattagctgcagcaaggacagcagtccttgcattcagtgatgtagtgataccagccttggcaatgctaacagtctgttgttccataacttcatgtatagatgtacgatca

SEQ ID NO: 10 - DNA, 121 bp, Pisum sativum
  source 1..121: mol_type = genomic DNA
ataccatgcaggattagctgcagcaaggacagcagtccttgcattcagtgatgtagtgatcccagccttggcaatgctaacagtctgttgttccataacttcatgtatagatgtacgatca

SEQ ID NO: 11 - DNA, 102 bp, Pisum sativum
  source 1..102: mol_type = genomic DNA
gtccctaatgctgcttatgctggtggtggcccaaggagttcatggcccgcacaggctccctctggctatggctctatgggttatggaaacactgctccttgg

SEQ ID NO: 12 - DNA, 102 bp, Pisum sativum
  source 1..102: mol_type = genomic DNA
gtccctaatgctgcttatgctggtggtggcccaaggagttcatggcccgctcaggctccctctggctatggctctatgggttatggaaacactgctccttgg

SEQ ID NO: 13 - DNA, 121 bp, Pisum sativum
  source 1..121: mol_type = genomic DNA
aggtggtgtttctgttttgtgttctttacttggtccttttacttcatatgctgttggttctgaagttattggtattcttgttagtttgacacttgattctgaatccaaaaagaatcttatg

SEQ ID NO: 14 - DNA, 121 bp, Pisum sativum
  source 1..121: mol_type = genomic DNA
aggtggtgtttctgttttgtgttctttacttggtccttttacttcatatgctgttggttccgaagttattggtattcttgttagtttgacacttgattctgaatccaaaaagaatcttatg

SEQ ID NO: 15 - DNA, 121 bp, Pisum sativum
  source 1..121: mol_type = genomic DNA
acatataatagcacgtcgaagatcttcatcgtccttactacagagcacttgcacatattgtataaggtttggaaacatctctttttccgttgttgatgacaacggaaaaagagacttttgt

SEQ ID NO: 16 - DNA, 121 bp, Pisum sativum; identical to SEQ ID NO: 15
  source 1..121: mol_type = genomic DNA

SEQ ID NO: 17 - DNA, 122 bp, Pisum sativum
  source 1..122: mol_type = genomic DNA
ggtttgcaagggacacaacatttggaagaaggcacttgagcatgtgggctcagtcacctattttgttatggattgtaagggaacttttgttacataaaattaatcatacacattaattaaat

SEQ ID NO: 18 - DNA, 122 bp, Pisum sativum
  source 1..122: mol_type = genomic DNA
ggtttgcaagggacacaacatttggaagaaggcacttgagcatgtgggctcagtgacctattttgttatggattgtaagggaacttttgttacataaaattaatcatacacattaattaaat

SEQ ID NO: 19 - DNA, 121 bp, Pisum sativum
  source 1..121: mol_type = genomic DNA
tacatcagtttgagaaagttacagcagaactcacaactcaagaagaaacttgcaatttgttatatcaaccggaatttcgccaacgaggtttaagttgctcaaatccagcaattcaagcagc

SEQ ID NO: 20 - DNA, 121 bp, Pisum sativum
  source 1..121: mol_type = genomic DNA
tacatcagtttgagaaagttacagcagaactcacaactcaagaagaaacttgcaatttgtgatatcaaccggaatttcgccaacgaggtttaagttgctcaaatccagcaattcaagcagc

SEQ ID NO: 21 - DNA, 121 bp, Pisum sativum
  source 1..121: mol_type = genomic DNA
cattacctcacttgaccaagccttcaaccaagcaaagaagcgtagtcaaaaagtttgtggagttataatatcaaacccttcaaaccctaccggaaaattcttaaatcgggaaacactactt

SEQ ID NO: 22 - DNA, 121 bp, Pisum sativum
  source 1..121: mol_type = genomic DNA
cattacctcacttgaccaagccttcaaccaagcaaagaagcgtagtcaaaaagtttgtggggttataatatcaaacccttcaaaccctaccggaaaattcttaaatcgggaaacactactt

(19) United States
(12) Patent Application Publication
(10) Pub. No.: US 2023/0255173 A1
(43) Pub. Date: Aug. 17, 2023

(54) HIGH PROTEIN PEA CONCENTRATES AND FOOD PRODUCTS MADE THEREOF

(71) Applicant: Equi-nom Ltd., Kibbutz Givat Brenner (IL)

(72) Inventors: Avinoam Lerer, Rehovot (IL); Inna Osmolovsky, Rehovot (IL); Sigal Meirovitch, Tel Aviv (IL); Oswald Crasta, Lubbock, TX (US); Gil Shalev, Ramot Meir (IL); Noa Palevsky, Rehovot (IL); Menachem Sklarz, Beer Sheva (IL); Avichai Amrad, Kfar Bilu (IL); Iftach Farber, Kibbutz Naan (IL)

(73) Assignee: Equi-nom Ltd., Kibbutz Givat Brenner (IL)

(21) Appl. No.: 18/191,443

(22) Filed: Mar. 28, 2023

Related U.S. Application Data
(63) Continuation of application No. PCT/IL2022/050911, filed on Aug. 19, 2022.
(60) Provisional application No. 63/234,782, filed on Aug. 19, 2021.

(51) Int. Cl. A01H 6/54; A23L 11/60; A01H 1/04; A01H 5/10; A23J 3/22

(52) U.S. Cl. CPC A01H 6/546; A01H 1/04; A01H 5/10; A23J 3/227; A23L 11/60

(57) ABSTRACT

Pea protein concentrates (PPCs) and food products made therefrom are provided. Disclosed PPCs comprise 65 wt% or more protein (dry base) and 2.2 wt% or less starch, and may be used for producing dairy analogues or protein-fortified beverages when having low gel firmness, or for producing texturized pea proteins for meat analogues when having high gel firmness. Further biochemical and compositional traits are used to adjust the PPCs to specific products and uses, and pea varieties that enable producing PPCs with high protein content and high yield are also provided.

### CROSS-REFERENCE TO RELATED APPLICATIONS

This application is a Continuation application of International Patent Application No. PCT/IL2022/050911, filed Aug. 19, 2022, which claims the benefit of U.S. Provisional Pat. Application No. 63/234,782 filed Aug. 19, 2021, which are hereby incorporated by reference.

### SEQUENCE LISTING STATEMENT

The instant application contains a Sequence Listing which has been submitted electronically in XML format and is hereby incorporated by reference in its entirety. The XML format copy, created on Oct. 3, 2022, is named P-607683-PC_SL.xml, and is 22 KB in size.

### BACKGROUND OF THE INVENTION

### 1. Technical Field

The present invention relates to the field of dairy products and meat replacements, and more particularly, to producing high protein pea concentrates.

### 2. Discussion of Related Art

Pea (Pisum sativum) is a cool season legume grown worldwide as a source of protein both for human food and animal feed. Economically, legumes represent the second most important family of crop plants, and dry pea currently ranks second only to common bean as the most widely grown grain legume in the world. Its primary production is in temperate regions. In 2018, its global production was 34.7 M tons.

Although pea is considered to be one of the world’s oldest domesticated crops, classical breeding methodology attempts done in order to increase protein level encountered some obstacles due to inferior agronomical traits such as low yield potential. Commercial dry pea varieties, which currently grown in France and Canada, have an average protein content of about 22%. The plant protein market is constantly challenged by the growing worldwide demand for non-GMO plant-based protein.

Dry fractionation of pulse proteins has been used as a sustainable method of plant protein extraction due to reduced use of energy and water (see, e.g., Moeller et al. 2021, From raw material to mildly refined ingredient - Linking structure to composition to understand fractionation processes, Journal of Food Engineering 291, 110321). However, since commercial dry fractionated PPC has relatively low protein content of <55% dry base (d.b) and substantial amount of starch 5%-8%, it is most commonly used for pet food, extruded snacks and baked goods applications where bulk is needed, and downstream processing can overcome the off flavors that associated with native PPC.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a dairy analogue or protein-fortified beverage comprising a pea protein concentrate (PPC), the PPC comprising 65 wt% or more protein (dry base), 2.5 wt% or less starch, 2.8 wt% or more sucrose, and a 1.3 [% from all sample protein in SDS PAGE lane] vicilin 1 and 2 or lower.

One aspect of the present invention provides a meat analogue comprising a PPC comprising 65 wt% or more protein, 2.2 wt% or less sucrose, 2.5 wt% or less starch and a 1.3 [% from all sample protein in SDS PAGE lane] vicilin 1 and 2 or higher.

One aspect of the present invention provides a PPC comprising 65 wt% or more protein (dry base) and 2.5 wt% or less starch.

One aspect of the present invention provides pea plant, cells or parts thereof configured, modified or selected to have specific biochemical composition and physical seed characteristics for high purity and efficiency of dry-fractionated air-classification protein products, as disclosed herein.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout. The patent or application file contains at least one drawing executed in color. Copies of this patent or patent application publication with color drawing(s) will be provided by the Office upon request and payment of the necessary fee. In the accompanying drawings:

FIG. 1 is a high-level schematic overview block diagram of pea protein products, according to some embodiments of the invention.

FIGS. 2A and 2B illustrate the relation of high gelation firmness to the parameters of vicilin and sucrose content, respectively, according to some embodiments of the invention.

FIGS. 3A and 3B provide images that illustrate the more uniform and delicate texture of PPC from disclosed pea varieties compared to prior art samples of PPC and PPI mixtures.

FIGS. 4A and 4B provide a relation between the theoretical light fraction protein yield and the content of the vicilin 6 protein component.

FIG. 4C provides a relation between the theoretical light fraction protein yield and the starch granule size.

FIG. 4D provides a relation between the gel firmness and the sucrose content (see also FIG. 2A).

FIG. 5 is a high-level schematic illustration of pea chromosomes with indications of the markers’ loci, according to some embodiments of the invention.

FIGS. 6A-6C present experimental results indicating the higher protein content and varying protein composition traits in pea varieties with the disclosed marker cassettes, according to some embodiments of the invention.

FIG. 7 is a high-level schematic illustration of the breeding method, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Embodiments of the present invention provide efficient and economical methods and mechanisms for producing high protein pea concentrates and thereby provide improvements to the technological field of dairy products and meat replacements. Pea protein concentrates (PPCs) and food products made therefrom are provided. Disclosed PPCs comprise 65 wt% or more protein (dry base) and 2.2 wt% or less starch, and may be used for producing dairy analogues or protein-fortified beverages when having low gelation firmness, or for producing meat analogues when having high gelation firmness. Further biochemical and compositional traits are used to adjust the PPCs to specific products and uses, and pea varieties that enable producing PPCs with high protein content and high yield are also provided.

The following application discloses processing methods and appropriate pea varieties for producing improved and new non-GMO (genetically modified organisms) pea protein products. Pea varieties, developed using computer-assisted breeding programs to have high protein content, modified biochemical compositions and other specific seed traits are used to enable the production, as well as achieve higher protein yield and purity, of dry-fractionated air-classified protein products which are optimized to yield specified functional traits of produced pea protein concentrates (PPCs), produce low-gelation and high-gelation PPCs, enable high moisture extrusion of the PPC to produce meat analogues, and improve the quality of low-moisture texturized pea proteins.

In various embodiments, correlations are provided between the biochemical composition of the PPCs and functional traits of resulting food products, high moisture extrusion of dry-fractionated PPC for meat analogues is demonstrated, and the yellow pea varieties with specific biochemical composition and physical seed characteristics for high purity and efficiency of dry-fractionated air-classification protein products are disclosed, specifically pea varieties which enable producing PPC by dry-fractionation with increased protein purity (e.g., up to 75% in contrast to prior art 55% on dry basis) and significantly improved PPC yield (e.g., up to 30% or more, in contrast to prior art 20%-25% at most) - resulting in new available products and superior product quality. Advantageously, disclosed PPC, produced as disclosed from disclosed pea varieties, reaches between 65% and 75% protein (dry matter) and exhibits a yield between 25% and 32%, in contrast to prior art PPC having up to 55% protein (dry matter) and up to 25% yield.

Disclosed dry fractionated pea protein concentrate provides a sustainable, functional, clean-label and cost-effective alternative to pea protein isolates (PPI) and may be used as a novel ingredient possessing high protein density of 65-75% (on dry base) and a wide range of functionalities, compatible for food applications where commercial pea protein concentrate (PPC) is incompatible due to, e.g., lower protein content, partial functionality, or inappropriate taste profile.

Disclosed dry fractionated pea protein concentrate has a macro-nutrient content and specific biochemical compositions, which offer a wide range of functional properties, compatible for different food applications. Disclosed concentrates thus offer high protein density with 65%-75% protein (dry base), low starch content <2.5%, and negligible amount of sodium <5 mg per 100 g. Furthermore, disclosed dry fractionated pea protein concentrate contains variable levels of Vicilin 1,2 (from 0.02 to 4.5) and variable levels of sucrose 1.8-3.6 g/100 g, providing a range of functionalities for new food applications as an alternative to PPI.

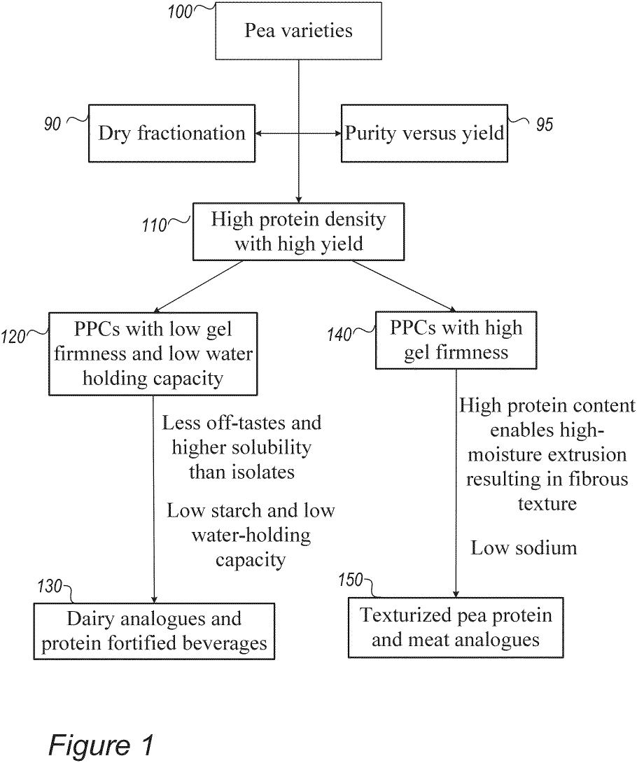

FIG. 1 is a high-level schematic overview block diagram of pea protein products, according to some embodiments of the invention. Pea varieties 100 disclosed herein (overall 144 pea varieties were tested herein) were processed in a dry fractionation process 90 which breaks down the pea seeds into component particles and applies air classification to sort out the light fraction of high-protein particles. Inherent in the classification process is a tradeoff 95 between purity (percentage of protein in the resulting concentrate, e.g., the light fraction) and yield (percentage of concentrate from the weight of dehulled pea grains) - the higher the required protein concentration the lower the yield. While typical prior art concentrates reach 50%, 55% or at most 60% protein content (dry base) at reasonably feasible yields, disclosed pea varieties 100 enable reaching higher protein content (e.g., 65%, 68%, 70%, 75%, intermediate or even higher content, dry base) while maintaining high process yield in disclosed processed pea concentrate 110. It is further noted that dry fractionation process 90 has inherent advantages over the wet fractionation process that is used to produce pea protein isolate with higher protein content, namely - dry fractionation is a much simpler and cheaper process, requiring significantly less energy and changing the biochemical composition of the proteins and other seed components much less than wet fractionation.

Moreover, pea varieties 100 disclosed herein enable further characterization and determination of other characteristics of disclosed concentrate 110 that relate to required characteristics of products made of disclosed concentrate 110. For example, certain varieties may yield pea protein concentrates (PPCs) 120 with low gel firmness and low water holding capacity, which is appropriate for producing dairy analogues and protein fortified beverages 130 therefrom, such as less off-tastes and high solubility than pea protein isolates, as well as a low sodium and starch content which are appropriate for use in dairy analogues. In another example, certain varieties may be used to reach PPCs 140 that are characterized by a high-gel firmness, which are appropriate for producing texturized pea proteins and meat analogues 150 therefrom, such as high protein content that enables low moisture and high-moisture extrusion resulting in anisotropic fibrous texture, and low sodium content, which are appropriate for use in meat analogues. See O′Kane 2004 (Molecular characterization and heat-induced gelation of pea vicilin and legumin, PhD thesis, Wageningen University, The Netherlands) for an overview of gelation processes.

Specifically, dairy analogues or protein-fortified beverages are disclosed, which comprise a pea protein concentrate (PPC) that comprises 65 wt%, 68 wt% or more protein (dry base), 2.2 wt% or less starch, 2.8 wt% or more sucrose, and a 1.3 protein percent fraction (protein percent from the whole protein volume in a lane of SDS-PAGE gel) of vicilin 1 and 2 or lower. The PPC may have a gelation firmness of 0.8 N or less, a high solubility of 75%PDI or more and a low water holding capacity (<0.6 gH₂O/gPPC). Corresponding pea protein concentrate for dairy analogues or protein-fortified beverages are likewise provided.

Disclosed dairy analogues and protein fortified beverages may include milk analogues, plant-based beverages, sports nutrition, ice cream, protein bars and protein fortified baked goods, etc., wherein disclosed PPCs are characterized by the following functional properties which are highly compatible for milk analogues.

Disclosed PPCs provide better taste, specifically with significantly less bitter, beany and grassy off notes to milk analogues and dairy applications by allowing up to 25% reduction in protein powder inclusion rate compared to commercial PPC with lower protein density. It is noted that the high protein content both reduces the content of other components with potentially unwanted taste, as well as requires less or no additions of other protein sources - avoiding unwanted taste introduction from these. Furthermore, due to the nature of dry fractionation process, disclosed PPC provides a fresh and sweet, and cleaner taste profile without cooked and umami flavors that are often associated with PPI due to its more intensive production process.

Disclosed PPCs also provide better solubility and mouthfeel, as it is significantly more soluble compared to other pea proteins such as PPI. PPI extraction process uses iso electric precipitation often results in poor solubility at 20-60 PDI%, while disclosed PPC offers significantly higher solubility at 65-90% PDI without using hydrolysis, therefore offering better mouthfeel with less aggregates compared to pea isolates.

Disclosed PPCs further provide improved texture and texture stability over shelf life, and their low starch content (<2.5 wt%) provides high compatibility for beverage applications where low viscosity and gelation are most needed, as the effect of gelatinization on viscosity is negligible at this concentration. Furthermore, the native form of protein in disclosed PPCs provides very low water holding (0.4-0.7 g water/1 g PPC), while protein isolates have significantly higher water holding, ranging from 1.5 g-4 g water/1 g PPI, by using different manufacturing processes. Therefore, disclosed PPCs for beverages provides “inert like” functional properties - including very low viscosity, high solubility and clean taste, allowing better control over the texture and taste of plant-based beverages and dairy analogues.

Additionally, meat analogues are disclosed, which comprise a pea protein concentrate (PPC) that comprises 65 wt%, 68 wt% or more protein (dry base), 2.2 wt% or less sucrose, and a 1.3% protein fraction of vicilin 1 and 2 or higher, and a sodium content of 5 mg/100 g or less. The PPC may have a gel firmness of 0.8 N or more, solubility of 65%PDI or more and may be used to produce the meat analogues by high-moisture extrusion (HME) or low moisture extrusion and/or without any pea protein isolate (PPI). Corresponding pea protein concentrates for meat analogues are likewise provided.

In certain embodiments, disclosed PPC may be used low moisture extrusion to yield products with 65% protein or more, without addition of PPI, or with addition of up to 20% PPI (in contrast to prior art practice of adding 50% PPI). In certain embodiments, disclosed PPC may be used high moisture extrusion to yield products with 65% protein or more, without addition of PPI, or with addition of up to 20% PPI (in contrast to prior art practice of adding 50% PPI, using PPC with high protein content of 68%, 70% or more enables the low inclusion rate of PPI).

Disclosed meat analogues may include texturized proteins, meat analogues, baked goods, plant-based puddings, and spreadable cheeses, etc., wherein disclosed PPCs are characterized by the following functional properties such as high gelation and fiber formation at high-moisture extrusion, which are highly compatible for meat analogues.

Disclosed PPCs provide clean label texturized protein formulations for both low moisture and high moisture extrusion contains, which may have between 55-70% protein (dry base), 10-18% insoluble dietary fibers in variable amount of starch, in order to provide the right texture and functionality.

The inventors note that while disclosed PPCs may be used as single or main protein component for producing meat analogues, in the prior art reaching a similar protein to fiber ratio requires an inclusion rate of 50%- 90% PPI in the formulation and addition of PPC, legume flours, fibers and starches based on the required texture, functionality and nutritional values - which are disadvantageous with respect to process complexity, cost, controllability and resulting off tastes. In contrast, disclosed PPCs provide a complete package of protein and fibers in one product, compatible for low moisture and high moisture extrusion applications, providing 65-75% protein and 12-18% fibers.

Moreover, disclosed PPCs provide a significant reduction in sodium, as it has much lower sodium levels than PPI that is used for most texturized proteins. For example, use of disclosed PPCs may reduce the sodium content in a burger analogue patty by up to 250 mg sodium per serving 4Oz (113 g), as disclosed PPCs may yield texturized pea protein (TPP) that include less than 5 mg/100 g sodium, in contrast to commercial PPI that contains 500-1200 mg sodium/100 g (e.g., commercial TTP having 70% protein may include 80% PPI and 20% pea flour). It is noted that the inclusion rate of TPP in burger analogue is typically between 20-30%, therefore TPP with 750 mg per 100 g, provides 750*0.25=187 mg sodium per 100 g, and a 113 g patty would correspondingly contain 187*1.13=211 mg sodium per 113 g patty, originated in TPP only. While typical meat analogue patties contain between 170-254 mg/100 g sodium, disclosed PPC contributes merely 1-2 mg/100 g sodium to meat analogue patties - leaving a large room to adjust the amount to sodium in the final product.

Advantageously, disclosed PPCs provide these functional properties and protein density required for high-moisture extrusion and are hence compatible for HMMA (high moisture extrusion meat analogue), in contrast to commercial pea protein concentrates that are incompatible for HMMA due to low protein density and functionality. Hence, disclosed PPCs offer a clean-label, sustainable and cost-effective alternatives to PPI.

Table 1 illustrates that multiple disclosed pea varieties were processed to yield disclosed PPCs with protein percentage above 65%, reaching above 70%, with high yields of 15% to almost 30%, in contrast to prior art varieties for which dry fractionated concentrates with high protein levels are not achievable commercially as these varieties provide protein concentration below 65%, and require a drastic reduction in yield (typically below 10%) in order to reach 65% protein content. Indicated alternatively, the protein separation efficiency (PSE) in dry fractionation of disclosed varieties is between 30-60% while the PSE of prior art commercial varieties is typically below 20% as protein purity of light fraction exceeding 60% protein on dry basis.

Table 1: Comparative protein purity and yield in disclosed varieties (numbered) versus prior art commercial varieties. Protein percentage is dry base (d.b.). Normalized yield to 65% protein indicates that dry fractionation is commercially not appropriate for producing high protein PPC from the commercial varieties.

$$\begin{matrix}\% P P C y i e l d a t 65 \% p r o t e i n \left(d . b\right) = \\ \frac{\left(\% P r o t e i n s e e d - \% P r o t e i n c o u r s e\right)}{\left(\% 65 - \% P r o t e i n c o u r s e\right)}\end{matrix}$$

| Variety | Dehulled seeds | Coarse fraction | Protein fraction | Protein fraction calculated | Protein fraction at 65% protein | Protein separation efficiency |
|---|---|---|---|---|---|---|
| | %protein | %protein | %protein | %yield | %yield | PSE (%) |
| * 6816 (2) | 31.2 | 16.0 | 68.2 | 29.1 | 31.0 | 63.7 |
| * 6768 (2) | 31.8 | 18.8 | 69.3 | 25.7 | 28.1 | 56.1 |
| * 8506 (1) | 32.4 | 17.9 | 70.4 | 27.6 | 30.8 | 60.0 |
| * 334 (2) | 31.6 | 18.9 | 67.9 | 25.9 | 27.5 | 55.7 |
| * 8510 (4) | 30.6 | 17.4 | 68.2 | 26.0 | 27.7 | 57.9 |
| * 6722 (1) | 32.0 | 18.9 | 71.1 | 25.1 | 28.4 | 55.8 |
| *French Pea -Ciacam | 22.5 | 14.5 | 55.4 | 19.6 | 15.8 | 48.2 |
| 8272 (1 and 4) | 33.9 | 23.2 | 72.2 | 21.8 | 25.5 | 46.4 |
| 8971 (4) | 34.4 | 24.3 | 70.1 | 22.1 | 24.9 | 45.0 |
| 6816 (2) | 33.5 | 22.5 | 70.9 | 19.6 | 22.4 | 43.3 |
| 6730 (2) | 33.7 | 23.7 | 70.2 | 21.4 | 24.1 | 44.6 |
| 6381 (1) | 33.3 | 23.7 | 71.2 | 20.2 | 23.2 | 43.2 |
| 8506 (1) | 33.8 | 24.9 | 72.9 | 15.1 | 18.3 | 28.6 |
| AAC Lacombe | 27.4 | 22.4 | 64.6 | 8.4 | 8.3 | 17.2 |
| CDC Amarillo | 29.0 | 20.3 | 64.0 | 17.1 | 16.7 | 36.2 |
| CDC Inca | 27.9 | 22.7 | 64.7 | 8.4 | 8.3 | 19.9 |
| CDC Meadow | 27.0 | 22.3 | 61.7 | 7.8 | 7.2 | 17.4 |
| CDC Spectrum | 29.4 | 24.0 | 65.1 | 9.0 | 9.0 | 16.7 |

*Produced at 3^{rd} party - IMPROVE SAS, Dury, France using the MPU ZPS 70, Hosokawa Alpine - see the detailed protocol below.

While most commercial references reached only 65.1% protein at 9% yield, suggesting that any further increase in classification parameters would not yield any significant increase in protein purity, disclosed varieties provided 70.2% protein with 23.3% yield on average.

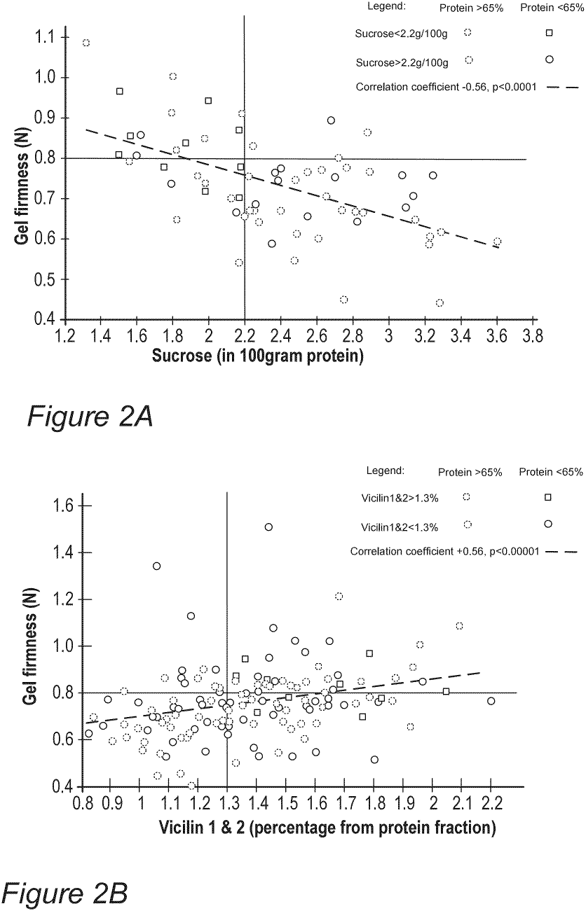

FIGS. 2A and 2B illustrate the relation of high gelation firmness to the parameters of vicilin and sucrose content, respectively, according to some embodiments of the invention. The graphs include multiple PPC products, charted with respect to their gelation firmness (in N) and vicilin/sucrose content. PPCs with gelation firmness above 0.8 N correspond roughly to vicilin 1&2 levels above 1.3% in the protein fraction and sucrose levels below 2.2 g/100 g protein.

It is noted that 60% of high protein PPCs with high gelation firmness (above 0.8N) were derived from disclosed varieties, while 40% of high protein PPCs with high gelation firmness (above 0.8 N) were derived from land-race varieties which were not used previously to produce PPC for meat analogues. Hence, the criterions for PPCs that are disclosed herein are also appropriate for detecting pea varieties which were hitherto not used but enable producing PPCs that can be used for meat analogues, beyond the disclosed varieties.

All of the high protein PPC having low gelation firmness (below 0.8 N) are made of disclosed pea varieties. It is noted that in both cases, low starch content contributes to the PPC characteristics - reducing water holding capacity and viscosity for low gelation PPC and avoiding gelatinization and enabling fiber formation for HMMA in high gelation PPC. In contrast, prior art PPCs with lower protein content include more starch (%Protein (d.b) in PPC X %Total starch in PPC R=-0.71, p=1.96·10⁻¹⁴, n=85), which in turn increases water holding capacity (WHC [gH₂O/gPPC] × %Total starch in PPC, R=0.45, p=1.78·10⁻⁵, n=85), (disadvantageous for dairy analogues) and is gelatinized under high moisture extrusion (may be disadvantageous for high moisture processing).

The following functional analysis data suggest new correlations between the biochemical composition of dry fractionated pea protein concentrates to their functional traits.

To measure gel firmness the samples were prepared as follows: 20% w/w PPC solution divided to 20 ml portions in 50 ml glass beakers, heated in a water bath to 85° C. for 1 hour, and later set overnight at 4° C. Prior to the analysis, the samples were taken out of the refrigerator until they reached 20° C. Texture analysis was performed with a TA-XT plusC Texture Analyzer (Texture Technologies Corp., Scarsdale, NY) using the P/0.5R cylindrical probe (12.7 mm diameter) set to 1 mm/sec penetration speed, to measure gel firmness at peak force.

Water/oil holding capacity was measured as follows: 10 ml water/oil were added to 1 g PPC sample in 50 ml centrifuge tube and homogenized at 21,500 rpm for 30 sec (CAT, Unidrive 1000D with 10 mm shaft), following by centrifugation at 1000 g for 15 min. the samples were than drained and the wet protein was weighted to measure the amount of water/oil absorbed per 1 g protein.

Protein composition was analyzed by a reducing sodium dodecyl sulphate polyacrylamide gel electrophoresis (SDS-PAGE) using a Criterion™ Vertical Electrophoresis Cell (BioRad Laboratories, Herculas, California, USA). Samples were prepared by mixing 6 µL of sample (1 g of flour from grounded seeds/ 40 ml NaOH, 3 mM) with 34 µL of sample buffer solution (24 µl of distilled water, 9 µl of 4X Laemmli sample buffer and 1 µl of β-mercaptoethanol). Samples were boiled for 5 min at 100° C. and then 10 µL of each sample were loaded on a 12% Criterion ™ TGX ™ Precast Gel (Bio-Rad Laboratories, Inc. Hercules, USA) and separated at 110 Volt for 90 min. Afterwards gels were stained with InstantBlue® Coomassie Protein Stain (Abcam) for 90 min and washed with distilled water for 90 min. Protein detection, analysis and documentation was performed by imaging system (gel Doc™EZ Imager, Bio-Rad).

Statistical analysis was carried out by analyzing the data (biochemical composition, physical analysis, functional traits) using Stepwise regression and Pearson Correlation using Python programming language.

The inventors have discovered several novel correlations which have not previously been reported in literature, between protein composition and functional traits, specifically regarding the characterization of sucrose functional traits - as summarized in Table 2 (and see the graphs in FIGS. 2A and 2B).

TABLE 2

Main correlations between PPC biochemical composition and functional traits
Gel firmness [N] n=144
Sucrose [g/100 g] (R= -0.56, p=0.000051)
Vicilin 1+2 (R = 0.56, p = 0.000004)

These correlations indicate that the functional traits of the PPC may be modified and improved using disclosed pea varieties in which protein composition was designed according to the discovered correlations

The production of palatable meat analogues using high moisture extrusion cooking is a complex process that depends on both the properties of the protein ingredients and the extrusion conditions (Osen et al., 2014 - High moisture extrusion cooking of pea protein isolates: raw material characteristics, extruder responses, and texture properties. Journal of Food Engineering, 127, and see also Osen et al. 2016 - High-Moisture Extrusion: Meat Analogues Fraunhofer Institute for Process Engineering and Packaging IVV, Freising, Germany). The inventors’ findings support the proposition that dry-fractionated pea protein concentrate containing more than 68% protein (on dry basis) can be a compatible raw material for the development of sustainable, cost-effective, fibrous-textured meat-analogues. It is noted that current high moisture texturized pea proteins are based on a mixture of 0%-40% pea flour and/or starch and/or fibers and 60-100% PPI that is more than double the cost of PPC. It is emphasized that currently no high moisture texturized PPC products (that are based solely on PPC, without additional treatments or additives) are available on the market or were ever described in the literature. Accordingly, disclosed PPC are new and unexpected in view of the current knowledge.

A high moisture extrusion cooking process (HMEC) was developed by using a lab-scale twin screw extruder with a cooling die (Brabender PTSE 12/36, Germany). The extruder configuration was a high-shear screw configuration with a screw diameter of 12 mm and a L/D ratio of 30. The cooling die geometry (l x h × w) was 300 × 9 × 20 [mm]. The extrusion parameters were: Feeding rate: 800 g / hour, screw speed: 571 rpm, total moisture content: 52% (dry wet base). The temperature profile was: 40, 80, 120, 140, 140, 75° C., under pressure of 23 bar.

Ingredients: The control sample of pea proteins for high moisture extrusion (68% protein % dry basis) prepared from 50% Pea protein isolate (80% protein dry basis, Pea Protein 80 FYPP-80), and 50% pea protein concentrate (55% protein dry basis, Anchor). The high moisture extrusion trials conducted at 57% total moisture content. The second high moisture extrudate was made solely from high protein PPC (disclosed PPC from disclosed pea varieties) containing 68% protein (dry basis %), at a total moisture content of 52% during high moisture extrusion. Both products were texturized under the same extrusion conditions (i.e., temp, pressure, speed, pressure).

The inventors have found the following experimental results. The nutritional values of the high moisture texturized proteins derived from disclosed pea varieties were better - with higher protein content and lower sodium - compared to commercially available alternative products, as indicated by Table 3.

TABLE 3

Nutritional values of disclosed vs. prior art TVPs

| Raw material % (dry base) | Disclosed PPC from disclosed pea varieties | Anchor PPC from prior art varieties | Control sample (PPI+PPC) |
|---|---|---|---|
| Protein | 32.0 | 24.0 | 31.0 |
| Moisture | 53 | 53 | 57 |
| Starch | <1 | 3.8 | 2.3 |
| Total dietary fibers | 5 | 7 | 4.5 |
| Ash | 2.5 | 2.5 | 2.35 |
| Sodium | <5 | <5 | 380 |

The organoleptic and physical characteristics were also found to be better in the texturized proteins derived from disclosed pea varieties. For textural evaluation, in order to describe the microstructure and textural characteristics of fibrous protein products made by high moisture extrusion, the method described by Osen et al., 2014 and Chen et al. 2010 (System parameters and product properties response of soybean protein extruded at wide moisture range. J. Food Eng. 96, 208-213) was used with slight modifications. A square shaped sample (20 × 20 mm) was cut using a knife blade (A/LKB probe), to 75% of its original thickness at a speed of 2 mm/s, and the cutting strength was recorded using a texture analyzer (TX.XT plusC, Stable Micro Systems, UK). Samples were evaluated vertically (longitudinal strength, Fl) and parallel (transverse strength, Ft) to the direction of extrudate outflow from the extruder. Values that are equal to or lower than one indicate uniform texture with low material anisotropy and without any fibrous texture. All determinations were performed with at least 12 replicates. According to Schutyser et al. 2015 (Dry fractionation for sustainable production of functional legume protein concentrates. Trends in Food Science & Technology 45(2):327-35), the Fl/Ft ratio of Chicken breast is 1.7, while Pea protein Isolate reaches 1.6 yields highly fibrous texture, and hamburger patty has a ratio of 1. Tables 4A and 4B provide the comparison of the Fl/Ft value among hamburger patty, PPC from disclosed pea varieties, the control sample, commercial PPC and chicken breast - indicating a desirable texture of disclosed PPC from disclosed pea varieties.

TABLE 4A

Correlations between biochemical composition and functionality to fiber formation (Fl/Ft - longitudinal to transverse strength ratio) in high moisture extrusion: High gel firmness, Vicilin 1,2 >1.3% of protein content, sucrose>2.2 g/100 g, low Legumin/Vicilin ratio

| Variety (QTL cassettes) | Protein % (dry base) | Fl/Ft** | Gel firmness [N] | vicilin 1+2 | sucrose g/100g | Legumin to vicilin ratio |
|---|---|---|---|---|---|---|
| 6816 (2) | 69.1 | 0.91 | 0.56 | 0.82 | 2.33 | 0.49 |
| 6730 (2) | 66.9 | 0.92 | 0.71 | 1.13 | 2.96 | 0.45 |
| 8272 (1 and 4) | 69.5 | 0.93 | 0.64 | 1.64 | 2.38 | 0.42 |
| 8971 (4) | 69.2 | 0.94 | 0.67 | 1.31 | 2.41 | 0.50 |
| 6381 (1) | 69.8 | 1.16 | 0.67 | 1.29 | 2.22 | 0.37 |
| 8506 (1) | 66.0 | 1.20 | 0.76 | 1.91 | 2.02 | 0.14 |
| 8506 (1) | 68.0 | 1.37 | 1.39 | 3.53 | 2.00 | 0.10 |

TABLE 4B

Fl/Ft value disclosed vs. prior art meat analogues

| | Hamburger patty | PPC from disclosed pea varieties | Control sample (50%PPI+50%PPC) | Commercial PPC | Chicken breast |
|---|---|---|---|---|---|
| Fl/Ft | 1 | 1.37 | 1.69 | -¹ | 1.7 |

¹Test failed as the commercial product was too sticky and had poor flowability. Additional experiments are being carried out to overcome these difficulties, which can be associated with low protein and hig starch and fiber content, to enable the comparison.

The following data suggests that PPC from disclosed pea varieties can be configured to have an anisotropic fibrous texture using high moisture extrusion cooking process, compatible for high moisture meat analogues. Initial tasting panel results suggest that PPC from disclosed pea varieties might be more compatible for chicken analogues compared to the control, due to softer and more elastic texture that highly resembles of chicken. Alternatively or complementarily, the biochemical compositions and/or other PPC or process traits may be modified to make PPC from disclosed pea varieties appropriate for a wide range of meat analogues.

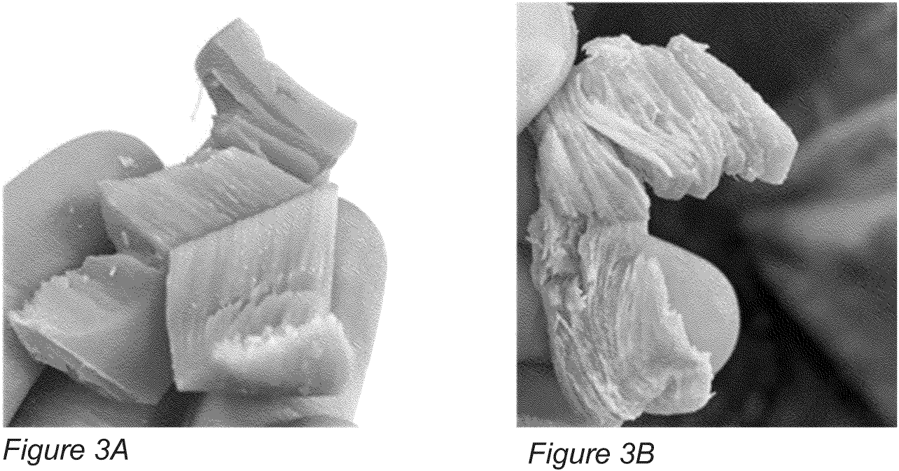

FIGS. 3A and 3B provide images that illustrate the more uniform and delicate texture of PPC from disclosed pea varieties compared to prior art samples of PPC and PPI mixtures. Specifically, FIG. 3A illustrates the finer fibrous texture of 100% PPC from disclosed pea varieties in contrast to FIG. 3B that illustrates a commercial mix of PPI and PPC (50%/50) (fibrous texture is not at all achievable with commercial PPC alone).

Concerning the taste profile, both samples presented typical subtle Umami flavor with no bitterness or beany off-flavors. With respect to density, no significant difference was found between high moisture texturized protein from disclosed pea varieties compared to control sample, measuring 1.18 g/ml (std 0.02).

Concerning the color, disclosed pea varieties yield lighter color of the texturized protein compared to the control, as seen in the images below. The lighter color may be more compatible with chicken analogue applications, in which light color textured protein is required, and moreover may offers greater color flexibility for a wider range of food applications.

The following discloses yellow pea varieties with specific biochemical composition and physical seed characteristics for high purity and efficiency of dry-fractionated air-classification protein products. Several routes to improve dry fractionation by plant breeding or pre-treatment techniques were previously reported in the literature. Plant breeding could focus on selection of varieties with larger starch granules, or tougher fibers or varieties with a lower seed hardness (Schutyser et al. 2015, and see also Dijkink et al. 2002, Milling properties of peas in relation to texture analysis. Part II. Effect of pea genotype.” Journal of Food Engineering 51(2): 105-111).

The following data suggest new biochemical and physical attributes which may be linked to higher protein separation efficiency (PSE) and pea protein concentrate yield (PPC yield) - as achievable by disclosed pea varieties.

The materials and methods were as follows. Dehulling - Abrasion dehulling conducted using seed whitener [(LSM, Schule®) equipped with 1.25*20 mm Sieve, 10 cm abrasion wheels], Following by hulls separation with a zig-zag classifier (MZM 1-40, Hosokawa-Alpine®) set to 23 m3/h(4.0 m.s-1) and an optical sorter (ZL1, Nage®) selection. Seed micronization - Dehulled seeds were coarsely milled by a knife mill (SM300, Retsch®) to have a size compatible with the feeding screw of the ZPS mill (200-500 µ). Pea flour micronization was done by ZPS Impact mill (50 ZPS + 50 ATP, Hosokawa Alpine®). Milling parameters: Nitrogen flow set to 60 m³/h, ZPS mill rotation speed at 14,000 rpm, ZPS milling selector wheel set to 3,500 rpm, feeding capacity of 6.6 Kg/h. Air classification - Following flour micronization, the product was air-classified (50 ATP, Hosokawa Alpine®). Classification parameters: Nitrogen flow set to 65 m³/h, classifier wheel rotation speed set to 12,000 rpm and a dosing screw set to 6.6 kg/h gravimetric capacity. A variety of the protocol which was used at IMPROVE SAS, Dury, France using the MPU ZPS 70, as denoted above in Tables 1 and 5 by asterisks (*), includes pea flour micronization done by ZPS Impact mill (70 ZPS + 70 ATP, Hosokawa Alpine®). Milling parameters: Nitrogen flow set to 100 m³/h, ZPS mill rotation speed at 13,000 rpm, ZPS milling selector wheel set to 3,800 rpm, feeding capacity of 10 Kg/h. Air classification - Following flour micronization, the product was air-classified (70 ATP, Hosokawa Alpine®). Classification parameters: Nitrogen flow set to 100 m³/h, classifier wheel rotation speed set to 9,000 rpm and a dosing screw set to 10 kg/h gravimetric capacity.

The analytical measurements were carried out as follows. Protein content - The protein contents were calculated based on the nitrogen content (N) using combustion method (Dumas) Rapid max N exceed (Elementar®) with a Nitrogen conversion factor of 6.25. The PDI (Protein Dispersibility Index) - measured based on AOCS official method BA 10b-09. The dry matter content was measured by a drying oven at 105° C. PrepASH system instrument (Precisa®). The particle size distribution was measured by laser diffraction, Helos-3000 (Sympatec Ltd.) equipped with a module for dry powder dispersion Quixel. A dispersion pressure of 4.5 bar was applied and the volume-weighted particle size distribution was measured. Protein separation efficiency (PSE) - The percentage of the total flour protein recovered in the light protein fraction was calculated as follows:

$$x f i n e = \frac{100 ∗ \left(\% P r o t e i n f l o u r - \% P r o t e i n c o u r s e\right)}{\left(\% P r o t e i n f i n e - \% P r o t e i n c o u r s e\right)}$$

Starch content in pea flour was measured by Total Starch Assay Kit (AA/AMG) (K-TSTA-100A 02/22, Megazyme International Ireland Ltd, Ireland) according to the assay procedure. AOAC Method 996.11. The starch composition in pea flour was measured by Amylose -Amylopectin enzymatic kit (K-AMYL 06/18, Megazyme International Ireland Ltd, Ireland). Sucrose content in pea flour was measured by Raffinose/Sucrose/D-Glucose Assay Kit (K-RAFGL 04/18, Megazyme International Ireland Ltd, Ireland) according to the assay procedure. The Statistical analysis was carried out by analyzing the data (biochemical composition, physical analysis, functional traits) using Stepwise regression and Pearson Correlation using Python programming language.

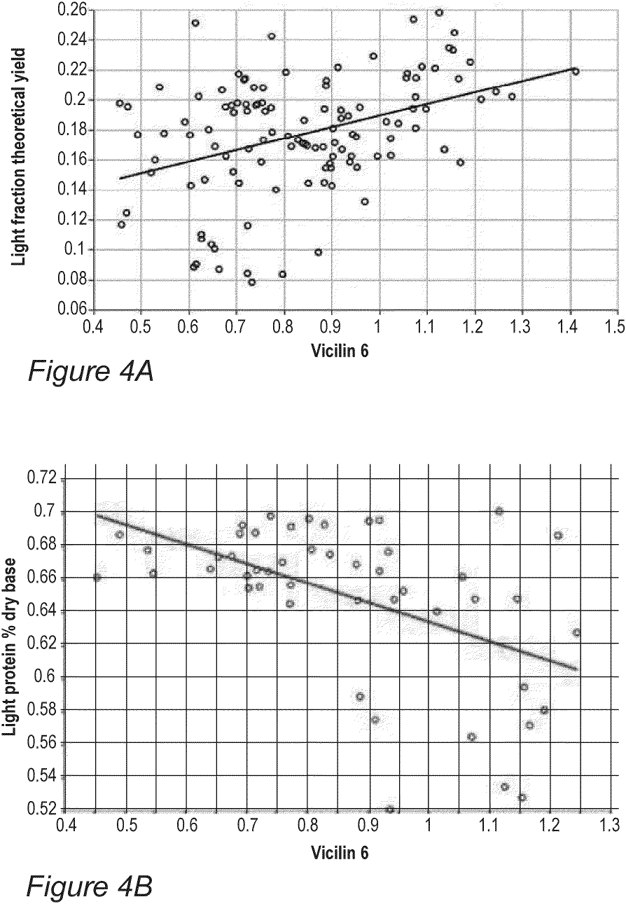

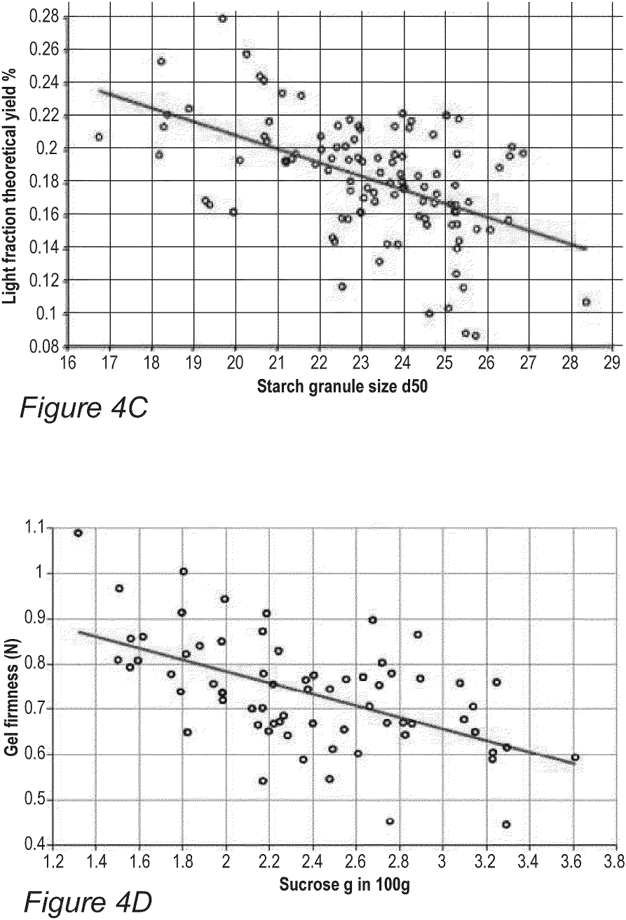

FIGS. 4A and 4B provide a relation between the theoretical light fraction protein yield and the content of the vicilin 6 protein component. The inventors have found that the following correlations suggest a novel approach to increase PPC yield in dry-fractionation processes, contributed by specific protein composition in the seed, specifically in relation to the vicilin 6 protein component. Specifically, the following novel correlations and related attributes (biochemical composition and physical seed characteristics) may be used to improve PSE (protein separation efficiency) and PPC (pea protein concentrate) yield:

Protein-composition related attributes (not known in the prior art):
Vicilin 6 - PPC yield (R = 0.40, p < 10⁻⁵, n=117) (FIG. 4A)
Vicilin 6 - PPC %protein dry base (-0.50. p=0.0003) (FIG. 4B)

Accordingly, in addition to high protein seed content (e.g., >29% dry base, contributing to >65% protein content dry base in the PPC), a high level of vicilin 6 also increases the fractionation process yield, e.g., a vicilin level above 1% of the total protein provides a yield of 20% or more.

FIG. 4C provides a relation between the theoretical light fraction protein yield and the starch granule size, FIG. 4D provides a relation between the gel firmness and the sucrose content (see also FIG. 2A). The inventors have found that the following correlations suggest a novel approach to increase PPC yield in dry-fractionation processes, contributed by specific protein composition in the seed, specifically in relation to starch and vicilin 6. Specifically, the following novel correlations and related attributes (biochemical composition and physical seed characteristics) may be used to improve PSE (protein separation efficiency) and PPC (pea protein concentrate) yield:

### Morphological Attributes

Starch granule size d50 - PPC yield (R=-0.51, p< 0.01) (FIG. 4C). In contrast to previous findings (from the literature), starch granule size was found to be negatively correlated with PSE and PPC yield.

The following data, listed in Table 5, illustrate that the disclosed PPC, produced as disclosed from disclosed pea varieties, reaches between 64% and 68% protein (dry matter) and exhibits a yield between 25% and 32%, in contrast to prior art PPC having up to 55% protein (dry matter) and up to 20% yield.

TABLE 5

Purity and yield of disclosed PPCs

| Disclosed pea seed variety (internal designation) | *PPC fractionation performance | | |
|---|---|---|---|
| | protein% (dry basis) | PPC yield% | |
| C6128 | 66.8 | 31.8 | High |
| C6126 | 65.8 | 31.3 | |
| C6184 | 67.5 | 30.9 | |
| C6185 | 67.8 | 30.9 | |
| C6129 | 65.7 | 30.4 | |
| C6127 | 68.0 | 28.8 | |
| C5553-6 | 63.1 | 27.6 | Medium |
| C5555-6 | 67.4 | 26.6 | |
| C5554-6 | 64.0 | 25.1 | |
| Commercial pea variety | 55.4 | 20.0 | Low |

*Produced at IMPROVE SAS, Dury, France, using ZPS 70 Hosokawa Alpine.

Disclosed (non-GMO) pea varieties may be configured, modified or selected, e.g., using the computer-assisted methods disclosed herein, to yield specific legumin to vicilin ratios, which are associated with the QTLs (quantitative trait loci) disclosed below. Accordingly, disclosed pea varieties may be optimized to enable and improve the air-classification process and the resulting products.

In the following, related and additional correlations are provided, which may provide further basis for optimization of disclosed pea varieties and of the production processes, yield products with improved qualities. It is noted that some of the results are being presently validated.

Additional findings suggest that disclosed pea varieties yield lower fiber and starch content, which further contribute to the efficiency of the dry-fractionated air-classification process and to the quality of the resulting products. For example, in contrast to commercial PPC products which typically include 6-8% starch (e.g., from Anchor, Ingredion, AGT brands), disclosed PPC from disclosed pea varieties may include between 1.8% and 3% starch, or in certain embodiments even lower values - providing a significant advantage.

Uses and pea plant cells of pea plants and parts thereof, which contain higher protein than current varieties, are provided. Phenotypic and genotypic analysis of many pea varieties was performed to derive markers for high protein and other phenotypic traits, and a breeding simulation was used to identify the most common and most stable markers. Following verification of trait stability over several generations, markers and marker cassettes were defined as being uniquely present in the developed pea lines. The resulting high protein pea lines can be used to enhance the nutritional values of pea in its various uses. Uses include processing the seeds to yield any of pea protein isolate, pea concentrate, a texturized product, a meat analog and/or commodity whole or split grains.

Uses and pea plant cells of pea plants and parts thereof, which contain higher protein than current varieties, are provided. Phenotypic and genotypic analysis of many pea varieties was performed to derive markers for high protein and other phenotypic traits, and a breeding simulation was used to identify the most common and most stable markers. Following verification of trait stability over several generations, markers and marker cassettes were defined as being uniquely present in the developed pea lines. The resulting high protein pea lines can be used to enhance the nutritional values of pea in its various uses. Uses include processing the seeds to yield any of pea protein isolate, pea concentrate, a texturized product, a meat analog or meat replacement and/or commodity whole or split grains. Certain embodiments comprise use of pea plants, parts thereof and/or pea seeds, which may be processed, as animal feed.

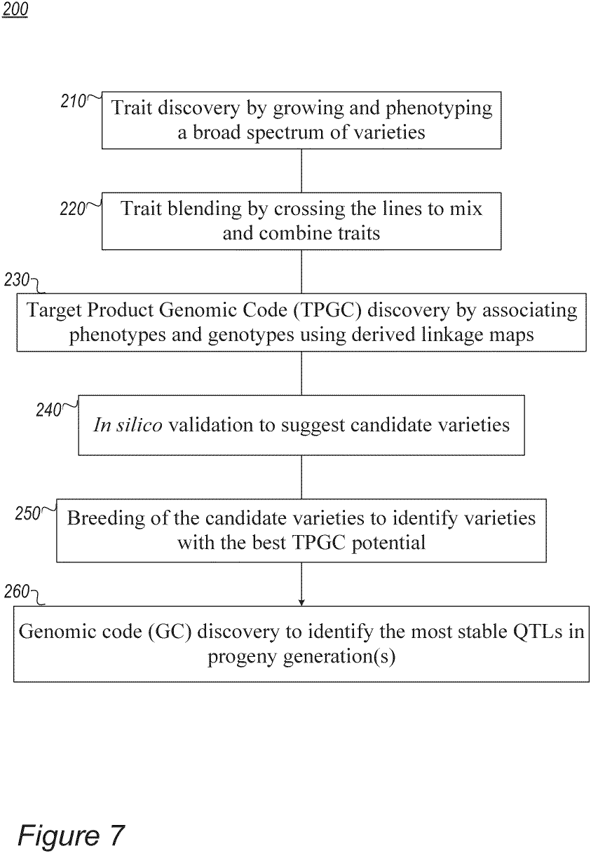

Various embodiments comprise pea cells and uses of pea plants or part(s) thereof that have high protein content and comprise a plurality of loci associated with a corresponding plurality quantitative trait loci (QTLs) having a corresponding plurality of nucleic acid genetic markers that are associated with a plurality of phenotypic traits of the pea plant. The phenotypic traits comprise a high protein content of the seeds of at least 25% and semi-leafless and powdery mildew resistance traits, and the plurality of QTLs and corresponding markers comprise at least two QTLs and corresponding markers - with details provided in Table 7 below. The methods used to develop and select the varieties are disclosed with respect to FIG. 7 below.

Various uses include processing the seeds to yield pea protein isolate and/or pea concentrate which provide the pea protein at different levels of concentration and with different amounts of additional compounds. The seeds may be processed into texturized products which may have mechanical properties in addition to their nutritional properties, e.g., texturized products may make a food product more firm or more elastic. The seeds may be processed into meat analogs to provide nutritional properties, chemical characteristics and similar look and feel (e.g., texture, flavor, appearance) as various types of meat. The seeds may be processed into commodity whole or split grains, possibly by drying or otherwise modifying the seeds.

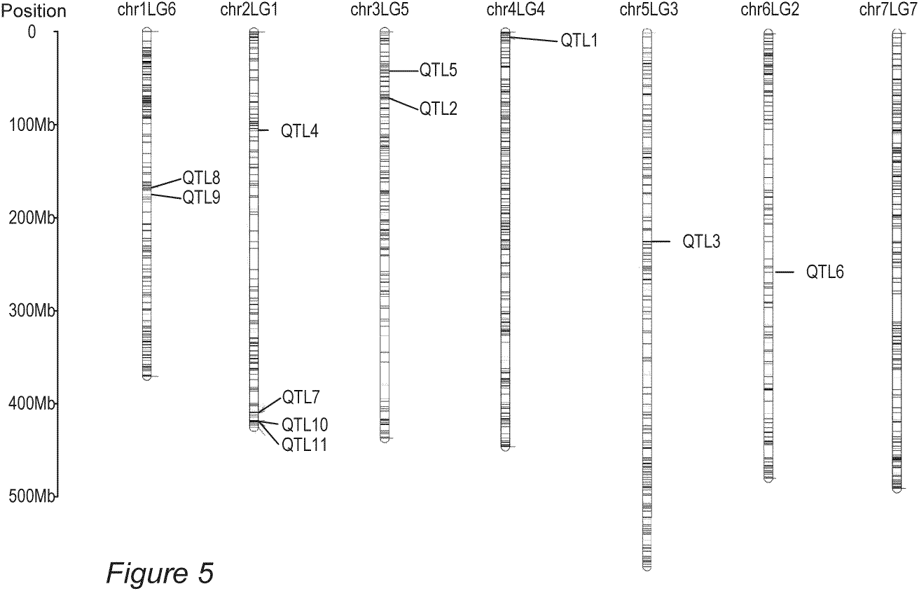

FIG. 5 is a high-level schematic illustration of pea chromosomes (and linkage groups -LGs) with indications of the markers’ loci (QTL number), according to some embodiments of the invention. FIG. 5 illustrates schematically the seven pea chromosomes with their banding patterns, and the marker locations indicated along them.

Table 7 provides the derived genetic markers, QTLs, corresponding traits and resulting marker cassettes, according to some embodiments of the invention.

Table 8 provides protein content and composition data for plant varieties with the marker cassettes, according to some embodiments of the invention - compared to control varieties.

TABLE 7

Genetic markers, QTLs, corresponding traits and marker cassettes with corresponding protein content and composition data

| QTL | Seq ID | Marker¹ | Chr/LG | Position | QTL P-value | Trait |
|---|---|---|---|---|---|---|
| 1 | 1,2 | 038887_23884_703 | chr4LG4 | 6445332 | 0.033 | Protein content |
| 2 | 3,4 | 017135_10651_5316 | chr3LG5 | 71669603 | 0.034 | Protein component |
| 3 | 5,6 | 050373_32960_3169 | chr5LG3 | 225883023 | 0.018 | Protein content |
| 4 | 7,8 | 029308_17474_1688 | chr2LG1 | 106604303 | 0.001 | Protein content |
| 5 | 9,10 | 044073_28004_2765 | chr3LG5 | 44420741 | 0.001 | Protein content |
| 6 | 11,12 | 07_32684348 | chr6LG2 | 259389351 | 0.013 | Protein component |
| 7 | 13,14 | 42662_26712_871 | chr2LG1 | 410200645 | Qualitative markers | Semi-Leafless |
| 8 | 15,16 | 044504_28363_461 | chr1LG6 | 167946502 | | Powdery mildew resistance |
| 9 | 17,18 | ER1 | chr1LG6 | 175515672 | | |
| 10 | 19,20 | 044835_28587_1878 | chr2LG1 | 419557580 | | Yellow cotyledon² |
| 11 | 21,22 | 044855_28602_1561 | chr2LG1 | 419560368 | | |

¹ The markers are as provided by Tayeh et al. 2015, except of 07_32684348 derived from independent RNA sequence analysis, and except of ER1 provided by Humphry et al. 2011.
² QTLs 10 and 11 for the yellow cotyledon trait are optional.

TABLE 7 continued

| QTL | | Allele | | Cassette (with respective QTLs)³ | | | |
|---|---|---|---|---|---|---|---|
| | Seq ID | 1 | 2 | 1 | 2 | 3 | 4 |
| 1 | 1,2 | T | G | GG/GT | TT/TG | | |
| 2 | 3,4 | A | G | AA/AG | GG/GA | | |
| 3 | 5,6 | A | G | | | AA/AG | |
| 4 | 7,8 | T | G | | | TT/TG | |
| 5 | 9,10 | A | C | | | | AA/AC |
| 6 | 11,12 | A | T | | | | AA/AT |
| 7 | 13,14 | T | C | TT/TC | TT/TC | TT/TC | TT/TC |
| 8 | 15,16 | T | C | | TT/TC | TT/TC | |
| 9 | 17,18 | C | G | GG/GC | | | |
| 10² | 19,20 | T | G | TT/TG | TT/TG | TT/TG | TT/TG |
| 11² | 21,22 | A | G | AA/AG | AA/AG | AA/AG | AA/AG |

² QTLs10 and 11 are optional for the respective cassettes.
³ For each QTL, the two Seq IDs represent the sequences for each allele; the zygosity (homozygous or heterozygous combination) is provided by the respective cassette entries.

TABLE 7 continued

| Cassette | QTLs | |
|---|---|---|
| | Obligatory | Optional |
| 1 | 1, 2, 7, 9 | 10, 11 |
| 2 | 1, 2, 7, 8 | 10, 11 |
| 3 | 3, 4, 7, 8 | 10, 11 |
| 4 | 5, 6, 7 | 10, 11 |

TABLE 8

Protein content and composition data for plant varieties with the marker cassettes

| Plants type (n) | Protein content (%) | | | Dunnett’s test 0.05 | | Protein composition (average, relative)¹ | | |
|---|---|---|---|---|---|---|---|---|
| | Average | Max | Min | Abs(Dif)-LSD | p-Value | Vicilin 6 | Vicilin 5 | P-value (ANOVA) |
| Cassette 1 (5) | 26.52 | 27.18 | 25.75 | 2.442 | <.0001 * | 1.7198 | | 0.0021 |
| Cassette 2 (12) | 26.88 | 28.59 | 25.84 | 2.134 | <0.0001* | | 1.10018 | 0.025 |
| Cassette 3 (5) | 26.76 | 27.44 | 26.37 | 1.898 | <0.0001 * | | | |
| Cassette 4 (2) | 26.4 | 26.81 | 25.98 | 1.381 | 0.0002* | | | |
| Control (5) | 23.41 | 24.56 | 22.43 | -1.22 | 1 | 1.19003 | 1.65058 | |

¹ Protein composition includes Vicilin traits 6 and 5 relating to QTL2, for cassettes 1 and 2, respectively. Further studies are underway.

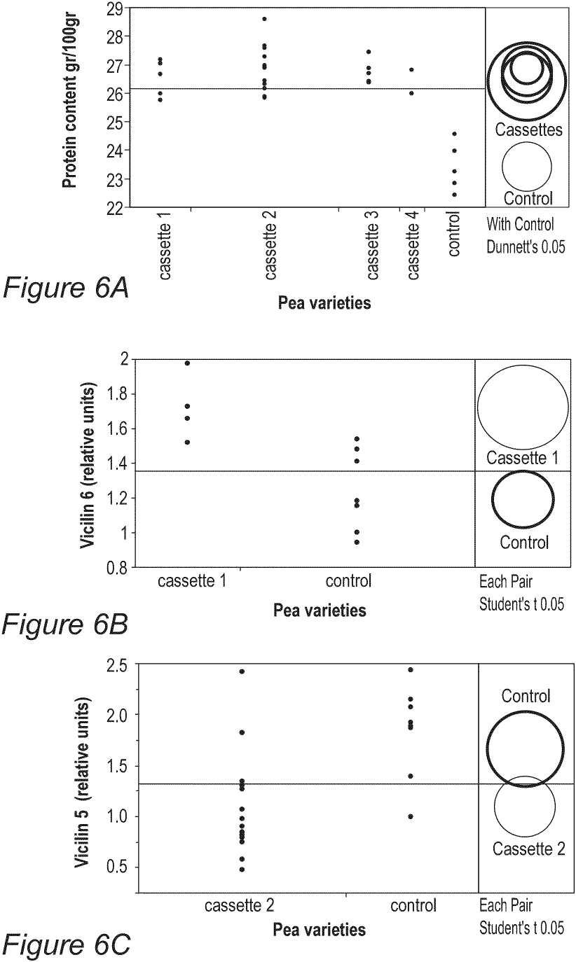

FIGS. 6A-6C present experimental results indicating the higher protein content and varying protein composition traits in pea varieties with the disclosed marker cassettes, according to some embodiments of the invention.

As indicated in Table 8 and illustrated in FIG. 6A, all four cassettes yield average protein content of at least 25% which is significantly distinct from prior art varieties that have lower protein content. FIG. 6A illustrates this significant difference graphically with respect to the multiple lines that were examined for each cassette (denoted by “n” in Table 8).

FIGS. 6B and 6C illustrate schematically the significant differences in protein composition of cassette 1 and cassette 2 varieties, with the former having significantly higher vicilin 6 content than the prior art varieties, and the latter having significantly lower vicilin 5 content than the prior art varieties. It is noted that Vicilin is one of the two major groups of storage proteins (Globulins) present in pea, together with Legumin (and Convicilin) it accounts for about 65-70% of the total protein content in the seed. Vicilin subunits are trimers, that form 6 subunits of 12-50 kDa. Vicilin 5 and 6 have the lowest molecular weight out of the subunits - 18 and 16 kDa respectively. All vicilin subunits are characterized by relatively high glycosylation, which contributes to their polarity and thus high water solubility and functionality. Accordingly, the inventors note that differences in protein composition may be related to the nutritional value and the processability of the resulting pea crop.

Disclosed QTLs comprise one or more of QTLs 1 to 11 with corresponding pairs of Seq IDs 1-22 that specify the alleles (with respective different SNP - Single Nucleotide Polymorphism - bases) of the respective markers that are linked to QTLs 1-11. It is noted that any of QTLs may be homozygous - having two identical alleles of the same Seq ID; or any of QTLs may be heterozygous having two different alleles with different Seq ID of each pair - as listed in Table 7 and below.

QTL 1, as used herein, refers to a polymorphic genetic locus linked to genetic marker 038887_23884_703 in pea linkage group 4 (LG4) on chromosome 4. The two alleles of marker 038887_238_703 at QTL 1 have the SNP bases “T” or “G”, respectively, at position 6445332 of LG4, as set forth, respectively, in the nucleic acid sequences of Seq IDs 1 and 2. In cassette 1, QTL 1 may be homozygous for allele 2 (Seq ID 2) or be heterozygous (Seq IDs 1 and 2); while in cassette 2, QTL 1 may be homozygous for allele 1 (Seq ID 1) or be heterozygous (Seq IDs 1 and 2).

Seq ID No. 1 (SNP base bold):

CTTTCTTCTGTATTTCCTTCTTTTCTTTTTCCTGGCCACCAAACAGCAGG
TTCATATTTCTCAGGAAACTTTTCAAGCATAACACCTAATAAAGGAAGAG
GATAAGCTTTATCAAGAGCCA

Seq ID No. 2 (SNP base bold):

CTTTCTTCTGTATTTCCTTCTTTTCTTTTTCCTGGCCACCAAACAGCAGG
TTCATATTTCGCAGGAAACTTTTCAAGCATAACACCTAATAAAGGAAGAG
GATAAGCTTTATCAAGAGCCA

QTL 2, as used herein, refers to a polymorphic genetic locus linked to genetic marker 017135_10651_5316 in pea linkage group 5 (LG5) on chromosome 3. The two alleles of marker 017135_10651_5316 at QTL 2 have the bases “A” or “G”, respectively, at position 71669603 of LG5, as set forth, respectively, in the nucleic acid sequences of Seq IDs 3 and 4. In cassette 1, QTL 2 may be homozygous for allele 1 (Seq ID 3) or be heterozygous (Seq IDs 3 and 4); while in cassette 2, QTL 2 may be homozygous for allele 2 (Seq ID 4) or be heterozygous (Seq IDs 3 and 4).

Seq ID No. 3 (SNP base bold):

TGAGATCACAGTTACTCAACATACAACTTAAATGAAATATAACGAATTAG
CATAAAACTCAAGAGGAGGGCATACATCTTCACCAATTGAAACAGCTTCA
GGGAAGAGCCCGTGAATGAGA

Seq ID No. 4 (SNP base bold):

TGAGATCACAGTTACTCAACATACAACTTAAATGAAATATAACGAATTAG
CATAAAACTCGAGAGGAGGGCATACATCTTCACCAATTGAAACAGCTTCA
GGGAAGAGCCCGTGAATGAGA

QTL 3, as used herein, refers to a polymorphic genetic locus linked to genetic marker 0503_32960_3169 in pea linkage group 3 (LG3) on chromosome 5. The two alleles of marker 050373_32960_3169 at QTL 3 have the bases “A” or “G”, respectively, at position 225883023 of LG3, as set forth, respectively, in the nucleic acid sequences of Seq IDs 5 and 6. In cassette 3, QTL 3 may be homozygous for allele 1 (Seq ID 5) or be heterozygous (Seq IDs 5 and 6).

Seq ID No. 5 (SNP base bold):

GTTTACATAAGATTAGAATGAATTGATCACTACTATACAGTTTTGAGAAA
TGAAATACACAAGGAATGCGTTATGTACGCAGAACAGGGAAAGGGAATCA
AGAATCGGTAGTGGAATCGAT

Seq ID No. 6 (SNP base bold):

GTTTACATAAGATTAGAATGAATTGATCACTACTATACAGTTTTGAGAAA
TGAAATACACGAGGAATGCGTTATGTACGCAGAACAGGGAAAGGGAATCA
AGAATCGGTAGTGGAATCGAT

QTL 4, as used herein, refers to a polymorphic genetic locus linked to genetic marker 029308_17474_1688 in pea linkage group 1 (LG1) on chromosome 2. The two alleles of marker 029308_17474_1688 at QTL 4 have the bases “T” or “G”, respectively, at position 106604303 of LG1, as set forth, respectively, in the nucleic acid sequences of Seq IDs 7 and 8. In cassette 3, QTL 4 may be homozygous for allele 1 (Seq ID 7) or be heterozygous (Seq IDs 7 and 8).

Seq ID No. 7 (SNP base bold):

TTTTTTGGTTCTTCTATAGACATATTCAACTAGTTTGTTTGCATCCATGG
TTCCTGTCACTGTTACTTTTCCTGTGCTAAACTCCGTCACTGCGGTTTGA
ACTCCTACAATAATCCATACA

Seq ID No. 8 (SNP base bold):

TTTTTTGGTTCTTCTATAGACATATTCAACTAGTTTGTTTGCATCCATGG
TTCCTGTCACGGTTACTTTTCCTGTGCTAAACTCCGTCACTGCGGTTTGA
ACTCCTACAATAATCCATACA

QTL 5, as used herein, refers to a polymorphic genetic locus linked to genetic marker 044073_28004_2765 in pea linkage group 5 (LG5) on chromosome 3. The two alleles of marker 044073_28004_2765 at QTL 5 have the bases “A” or “C”, respectively, at position 44420741 of LG5, as set forth, respectively, in the nucleic acid sequences of Seq IDs 9 and 10. In cassette 4, QTL 5 may be homozygous for allele 1 (Seq ID 9) or be heterozygous (Seq IDs 9 and 10).

Seq ID No. 9 (SNP base bold):

ATACCATGCAGGATTAGCTGCAGCAAGGACAGCAGTCCTTGCATTCAGTG
ATGTAGTGATACCAGCCTTGGCAATGCTAACAGTCTGTTGTTCCATAACT
TCATGTATAGATGTACGATCA

Seq ID No. 10 (SNP base bold):

ATACCATGCAGGATTAGCTGCAGCAAGGACAGCAGTCCTTGCATTCAGTG
ATGTAGTGATCCCAGCCTTGGCAATGCTAACAGTCTGTTGTTCCATAACT
TCATGTATAGATGTACGATCA

QTL 6, as used herein, refers to a polymorphic genetic locus linked to genetic marker 07_32684348 in pea linkage group 2 (LG2) on chromosome 6. The two alleles of marker 07_32684348 at QTL 6 have the bases “A” or “T”, respectively, at position 259389351 of LG2, as set forth, respectively, in the nucleic acid sequences of Seq IDs 11 and 12. In cassette 4, QTL 6 may be homozygous for allele 1 (Seq ID 11) or be heterozygous (Seq IDs 11 and 12).

Seq ID No. 11 (SNP base bold):

GTCCCTAATGCTGCTTATGCTGGTGGTGGCCCAAGGAGTTCATGGCCCGC
ACAGGCTCCCTCTGGCTATGGCTCTATGGGTTATGGAAACACTGCTCCTT
GG

Seq ID No. 12 (SNP base bold):

GTCCCTAATGCTGCTTATGCTGGTGGTGGCCCAAGGAGTTCATGGCCCGC
TCAGGCTCCCTCTGGCTATGGCTCTATGGGTTATGGAAACACTGCTCCTT
GG

QTL 7, as used herein, refers to a polymorphic genetic locus linked to genetic marker 42662_26712_871 in pea linkage group 1 (LG1) on chromosome 2. The two alleles of marker 42662_26712_871 at QTL 7 have the bases “T” or “C”, respectively, at position 410200645 of LG1, as set forth, respectively, in the nucleic acid sequences of Seq IDs 13 and 14. In cassettes 1 to 4, QTL 7 may be homozygous for allele 1 (Seq ID 13) or be heterozygous (Seq IDs 13 and 14).

Seq ID No. 13 (SNP base bold):

AGGTGGTGTTTCTGTTTTGTGTTCTTTACTTGGTCCTTTTACTTCATATG
CTGTTGGTTCTGAAGTTATTGGTATTCTTGTTAGTTTGACACTTGATTCT
GAATCCAAAAAGAATCTTATG

Seq ID No. 14 (SNP base bold):

AGGTGGTGTTTCTGTTTTGTGTTCTTTACTTGGTCCTTTTACTTCATATG
CTGTTGGTTCCGAAGTTATTGGTATTCTTGTTAGTTTGACACTTGATTCT
GAATCCAAAAAGAATCTTATG

QTL 8, as used herein, refers to a polymorphic genetic locus linked to genetic marker 044504_28363_461 in pea linkage group 6 (LG6) on chromosome 1. The two alleles of marker 044504_28363_461 at QTL 8 have the bases “T” or “C”, respectively, at position 167946502 of LG6, as set forth, respectively, in the nucleic acid sequences of Seq IDs 15 and 16. In cassettes 2 and 3, QTL 8 may be homozygous for allele 1 (Seq ID 15) or be heterozygous (Seq IDs 15 and 16).

Seq ID No. 15 (SNP base bold):

ACATATAATAGCACGTCGAAGATCTTCATCGTCCTTACTACAGAGCACTT
GCACATATTGTATAAGGTTTGGAAACATCTCTTTTTCCGTTGTTGATGAC
AACGGAAAAAGAGACTTTTGT

Seq ID No. 16 (SNP base bold):

ACATATAATAGCACGTCGAAGATCTTCATCGTCCTTACTACAGAGCACTT
GCACATATTGCATAAGGTTTGGAAACATCTCTTTTTCCGTTGTTGATGAC
AACGGAAAAAGAGACTTTTGT

QTL 9, as used herein, refers to a polymorphic genetic locus linked to genetic marker ER1 in pea linkage group 6 (LG6) on chromosome 1. The two alleles of marker ER1 at QTL 9 have the bases “C” or “G”, respectively, at position 175515672 of LG6, as set forth, respectively, in the nucleic acid sequences of Seq IDs 17 and 18. In cassette 1, QTL 9 may be homozygous for allele 2 (Seq ID 18) or be heterozygous (Seq IDs 17 and 18).

Seq ID No. 17 (SNP base bold):

GGTTTGCAAGGGACACAACATTTGGAAGAAGGCACTTGAGCATGTGGGCT
CAGTCACCTATTTTGTTATGGATTGTAAGGGAACTTTTGTTACATAAAAT
TAATCATACACATTAATTAAAT

Seq ID No. 18 (SNP base bold):

GGTTTGCAAGGGACACAACATTTGGAAGAAGGCACTTGAGCATGTGGGCT
CAGTGACCTATTTTGTTATGGATTGTAAGGGAACTTTTGTTACATAAAAT
TAATCATACACATTAATTAAAT

QTL 10, as used herein, refers to a polymorphic genetic locus linked to genetic marker 044835_28587_1878 in pea linkage group 1 (LG1) on chromosome 2. The two alleles of marker 044835_28587_1878 at QTL 10 have the bases “T” or “G”, respectively, at position 419557580 of LG1, as set forth, respectively, in the nucleic acid sequences of Seq IDs 19 and 20. In cassettes 1 to 4, QTL 10 may be homozygous for allele 1 (Seq ID 19) or be heterozygous (Seq IDs 19 and 20).

Seq ID No. 19 (SNP base bold):

TACATCAGTTTGAGAAAGTTACAGCAGAACTCACAACTCAAGAAGAAACT
TGCAATTTGTTATATCAACCGGAATTTCGCCAACGAGGTTTAAGTTGCTC
AAATCCAGCAATTCAAGCAGC

Seq ID No. 20 (SNP base bold):

TACATCAGTTTGAGAAAGTTACAGCAGAACTCACAACTCAAGAAGAAACT
TGCAATTTGTGATATCAACCGGAATTTCGCCAACGAGGTTTAAGTTGCTC
AAATCCAGCAATTCAAGCAGC

QTL 11, as used herein, refers to a polymorphic genetic locus linked to genetic marker 044855_28602_1561 in pea linkage group 1 (LG1) on chromosome 2. The two alleles of marker 044855_28602_1561 at QTL 11 have the bases “A” or “G”, respectively, at position 419560368 of LG1, as set forth, respectively, in the nucleic acid sequences of Seq IDs 21 and 22. In cassettes 1 to 4, QTL 11 may be homozygous for allele 1 (Seq ID 21) or be heterozygous (Seq IDs 21 and 22).

Seq ID No. 21 (SNP base bold):

CATTACCTCACTTGACCAAGCCTTCAACCAAGCAAAGAAGCGTAGTCAAA
AAGTTTGTGGAGTTATAATATCAAACCCTTCAAACCCTACCGGAAAATTC
TTAAATCGGGAAACACTACTT

Seq ID No. 22 (SNP base bold):

CATTACCTCACTTGACCAAGCCTTCAACCAAGCAAAGAAGCGTAGTCAAA
AAGTTTGTGGGGTTATAATATCAAACCCTTCAAACCCTACCGGAAAATTC
TTAAATCGGGAAACACTACTT

Disclosed pea plant having high protein content, or part(s) thereof are provided. The pea plant comprises a plurality of loci associated with a corresponding plurality of QTLs having a corresponding plurality of nucleic acid genetic markers that are associated with a plurality of phenotypic traits of the pea plant, wherein the phenotypic traits comprise a high protein content of the seeds of at least 25% and semi-leafless and powdery mildew resistance, and wherein the plurality of QTLs and corresponding markers comprise at least two QTLs and corresponding markers.

In certain embodiments, the QTL and marker associated with the high protein trait comprise QTL 1 with corresponding marker set forth in Seq. IDs 1 or 2; the QTL and marker associated with the semi-leafless trait comprise QTL 7 with corresponding markers set forth in Seq. IDs 13 or 14; and the QTL and marker associated with the powdery mildew resistance trait comprise QTL 9 with corresponding markers set forth in Seq. IDs 17 or 18.

In certain embodiments, the QTL and marker associated with the high protein trait comprise QTL 1 with corresponding marker set forth in Seq. IDs 1 or 2; the QTL and marker associated with the semi-leafless trait comprise QTL 7 with corresponding markers set forth in Seq. IDs 13 or 14; and the QTL and marker associated with the powdery mildew resistance trait comprise QTL 8 with corresponding markers set forth in Seq. IDs 15 or 16.

In certain embodiments, the pea plant or part thereof may further comprise a QTL and marker associated with a protein composition trait that comprise QTL 2 with corresponding marker set forth in Seq. IDs 3 or 4.

In certain embodiments, the QTL and marker associated with the high protein trait comprise QTL 3 with corresponding marker set forth in Seq. IDs 5 or 6; and QTL 4 with corresponding marker set forth in Seq. IDs 7 or 8; the QTL and marker associated with the semi-leafless trait comprise QTL 7 with corresponding markers set forth in Seq. IDs 13 or 14; and the QTL and marker associated with the powdery mildew resistance trait comprise QTL 8 with corresponding markers set forth in Seq. IDs 15 or 16.

In certain embodiments, the QTL and marker associated with the high protein trait comprise QTL 5 with corresponding marker set forth in Seq. IDs 9 or 10; and the QTL and marker associated with the semi-leafless trait comprise QTL 7 with corresponding markers set forth in Seq. IDs 13 or 14. The pea plant or part thereof may further comprise a QTL and marker associated with a protein composition trait that comprise QTL 6 with corresponding marker set forth in Seq. IDs 11 or 12.

In certain embodiments, the phenotypic traits further comprise a protein composition trait and/or a yellow cotyledon trait. In certain embodiments, the plurality of QTLs and corresponding markers comprise at least three QTLs and corresponding markers. In certain embodiments, the phenotypic traits further comprise a yellow cotyledon trait, for example, the QTLs and markers associated with the yellow cotyledon trait comprise QTL 10 with corresponding markers set forth in Seq. IDs 19 or 20; and/or QTL 11 with corresponding markers set forth in Seq. IDs 21 or 22.

In certain embodiments, the phenotypic traits comprise a high protein content of the seeds of at least 25%, or possibly a high protein content of the seeds of at least 29% on dry basis. In various embodiments, the plants may be hybrids and/or the plant parts may comprise any of: a seed, an endosperm, an ovule, pollen, cell, cell culture, tissue culture, plant organ, protoplast, meristem, embryo, or a combination thereof.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the plurality of QTLs and corresponding markers comprise at least three QTLs and corresponding markers, the QTL and marker associated with the semi-leafless trait comprise QTL 7 with corresponding markers set forth in Seq. IDs 13 or 14, and the pea plant or part thereof is homozygous with respect to Seq. ID 13 or heterozygous at QTL 7.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the plurality of QTLs and corresponding markers comprise at least three QTLs and corresponding markers, the QTL and marker associated with the semi-leafless trait comprise QTL 7 with corresponding markers set forth in Seq. IDs 13 or 14, and the pea plant cell is homozygous with respect to Seq. ID 13 or heterozygous at QTL 7.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant or part thereof comprise QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 9 with corresponding markers set forth in Seq. IDs 17 or 18, the pea plant or part thereof is homozygous with respect to Seq. ID 2 or heterozygous at QTL 1, the pea plant or part thereof is homozygous with respect to Seq. ID 3 or heterozygous at QTL 2, the pea plant or part thereof is homozygous with respect to Seq. ID 13 or heterozygous at QTL 7, and the pea plant or part thereof is homozygous with respect to Seq. ID 18 or heterozygous at QTL 9.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant or part thereof comprise QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant or part thereof is homozygous with respect to Seq. ID 1 or heterozygous at QTL 1, the pea plant or part thereof is homozygous with respect to Seq. ID 4 or heterozygous at QTL 2, the pea plant or part thereof is homozygous with respect to Seq. ID 13 or heterozygous at QTL 7, and the pea plant or part thereof is homozygous with respect to Seq. ID 15 or heterozygous at QTL 8.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 3 with corresponding marker set forth in Seq. IDs 5 or 6; and QTL 4 with corresponding marker set forth in Seq. IDs 7 or 8, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant or part thereof is homozygous with respect to Seq. ID 5 or heterozygous at QTL 3, the pea plant or part thereof is homozygous with respect to Seq. ID 7 or heterozygous at QTL 4, the pea plant or part thereof is homozygous with respect to Seq. ID 13 or heterozygous at QTL 7, and the pea plant or part thereof is homozygous with respect to Seq. ID 15 or heterozygous at QTL 8.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 5 with corresponding marker set forth in Seq. IDs 9 or 10, the pea plant or part thereof comprise QTL 6 with corresponding marker set forth in Seq. IDs 11 or 12, the QTL and marker associated with the semi-leafless trait comprise QTL 7 with corresponding markers set forth in Seq. IDs 13 or 14, the pea plant or part thereof is homozygous with respect to Seq. ID 9 or heterozygous at QTL 5, the pea plant or part thereof is homozygous with respect to Seq. ID 11 or heterozygous at QTL 6, and the pea plant or part thereof is homozygous with respect to Seq. ID 13 or heterozygous at QTL 7.

Some aspects of the present invention relate to uses of the pea plant or a part thereof, e.g., as textured vegetable products (TVPs), e.g., meat replacements, possibly having above 60% protein, above 65% protein or above 70% protein.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant obtained from said pea plant cell, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant cell comprises QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 9 with corresponding markers set forth in Seq. IDs 17 or 18, the pea plant cell is homozygous with respect to Seq. ID 2 or heterozygous at QTL 1, the pea plant cell is homozygous with respect to Seq. ID 3 or heterozygous at QTL 2, the pea plant cell is homozygous with respect to Seq. ID 13 or heterozygous at QTL 7, and the pea plant cell is homozygous with respect to Seq. ID 18 or heterozygous at QTL 9.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 1 with corresponding marker set forth in Seq. IDs 1 or 2, the pea plant cell comprises QTL 2 with corresponding marker set forth in Seq. IDs 3 or 4, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant cell is homozygous with respect to Seq. ID 1 or heterozygous at QTL 1, the pea plant cell is homozygous with respect to Seq. ID 4 or heterozygous at QTL 2, the pea plant cell is homozygous with respect to Seq. ID 13 or heterozygous at QTL 7, and the pea plant cell is homozygous with respect to Seq. ID 15 or heterozygous at QTL 8.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 3 with corresponding marker set forth in Seq. IDs 5 or 6; and QTL 4 with corresponding marker set forth in Seq. IDs 7 or 8, the QTL and marker associated with the semi-leafless trait comprise QTL 7 with corresponding markers set forth in Seq. IDs 13 or 14, the QTL and marker associated with the powdery mildew resistance trait comprise QTL 8 with corresponding markers set forth in Seq. IDs 15 or 16, the pea plant cell is homozygous with respect to Seq. ID 5 or heterozygous at QTL 3, the pea plant cell is homozygous with respect to Seq. ID 7 or heterozygous at QTL 4, the pea plant cell is homozygous with respect to Seq. ID 13 or heterozygous at QTL 7, and the pea plant cell is homozygous with respect to Seq. ID 15 or heterozygous at QTL 8.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 5 with corresponding marker set forth in Seq. IDs 9 or 10, the pea plant cell comprises QTL 6 with corresponding marker set forth in Seq. IDs 11 or 12, the QTL and marker associated with the semi-leafless trait comprise QTL 7 with corresponding markers set forth in Seq. IDs 13 or 14, the pea plant cell is homozygous with respect to Seq. ID 9 or heterozygous at QTL 5, the pea plant cell is homozygous with respect to Seq. ID 11 or heterozygous at QTL 6, and the pea plant cell is homozygous with respect to Seq. ID 13 or heterozygous at QTL 7.

FIG. 7 is a high-level schematic illustration of a breeding method 200, according to some embodiments of the invention. Breeding method 200 comprises stages of trait discovery by growing and phenotyping a broad spectrum of varieties (stage 210), trait blending by crossing the lines to mix and combine traits (stage 220), target Product Genomic Code (TPGC) discovery by associating phenotypes and genotypes using derived linkage maps (stage 230), in silico validation to suggest candidate varieties (stage 240), breeding of the candidate varieties to identify varieties with the best TPGC potential (stage 250) and genomic code (GC) discovery to identify the most stable QTLs in progeny generation(s) (stage 260), as explained in detail below.

In embodiments, pea lines were bred to reach high protein levels by collecting various pea lines worldwide, creating F2 linkage populations, applying intensive phenotyping and genotyping of thousands of pea lines, predicting of QTL’s affecting the protein level trait, and establishing unique marker combinations, termed “marker cassettes” herein, to characterize novel high protein level lines found by the method and not existing in commercial or natural lines.

The breeding methodology was based on algorithms for deriving the Target Product Genomic Code (TPGC) to associate (i) the Target Product (TP) being defined in advance based on market requirements and including a set of desired attributes (traits) that are available in natural genetic variations; and (ii) the Genomic Code (GC) comprising set(s) of genomic regions that include quantitative trait loci (QTLs) that affect and are linked to the TP traits. The algorithms may be configured to calculate multiple genomic interactions and to maximize the genomic potential of specific plants for the development of new varieties. The breeding program was constructed to derive the TPGC, and then by crossing and selfing to achieve a product which contains the specific GC that corresponds to the required TPs.

Certain embodiments of the breeding process comprise stages such as: (i) Trait Discovery, in which a broad spectrum of varieties from different geographies and worldwide sources are grown and phenotyped in order to discover new traits that can potentially be combined to create a new product; (ii) Trait Blend, in which a crossing cycle is carried out based on phenotypic assumption(s), in which the different traits are mixed and combined. Initial trait cycle(s) are followed by additional cycle(s) to create F2 (and possibly higher generations) population(s) that provide the basis for algorithmic analysis for constructing the TPGC; (iii) TPGC Discovery, in which the plant(s) are phenotyped and genotyped to produce linkage map(s), discovering the relevant QTLs and deriving the TPGC; (iv) several line validation stages over several years, in which pea lines based on millions of in silico calculated variations (and/or selections) are grown and are used to defined the initial varieties; (v) Trait TPGC Blend, in which accurate crossings are performed in order to calculate the most efficient way to reach the best TPGC. The crossings are performed after in silica selection from millions of combinations, and are based, at least on part on phenotype assumptions; and (vi) Consecutive algorithm-based GC discovery stage(s) applied to F2 (or higher generation) population(s) grown in additional cycle(s).

Defining the TP for high protein level pea varieties includes the development of high throughput methods for high protein level identification. Protein level was measured using the total Kjeldahl Nitrogen method (heating the sample with concentrated sulfuric acid and optionally a catalyst to oxidize the sample and liberate the reduced nitrogen as ammonium sulfate, followed by distillation) and total amino acid analysis after acid hydrolysis. In order to screen thousands of individuals every season, NIR (near infrared) analyzers were calibrated to measure total protein, total amino acid and moisture content using a wide spectrum of pea seeds compositional analysis. For protein composition, densitometry analysis of SDS PAGE (sodium dodecyl sulfate -polyacrylamide gel electrophoresis) was used to quantify pea two major storage proteins, legumin and vicilin. In addition, in order to breed for commercial pea protein varieties, phenotypic traits such as semi-leafless, yellow cotyledon and powdery mildew resistance were also included in the target product and as part of the TPGC. In various embodiments, TPGC includes combinations of unique traits (relating to high protein levels and to other phenotypic traits) that are associated with combinations of QTLs - yielding for high protein pea.

In the following non-limiting example of the process, Trait Discovery (i) was based on germplasm including four hundred different pea lines that were obtained from the gene banks around the world. Of these, fifty different lines were used for the Trait Blend stage (ii), with crosses executed based on the potential for enrichment of genomic diversity to create new complex(es) of traits for the high protein level as the initial step for the TP-directed breeding program for high protein level pea lines. The resulted F1 hybrids were later self-crossed to create F2 linkage populations that showed phenotypic segregation. The F2 population were then planted in two different environments for discovering the TPGC (iii) that includes high protein level traits. After screening 90,000 individuals, a set of ca. 3200 representatives was selected. The selected individuals F2 was massively phenotyped for high protein level, seed color, leaf type and powdery mildew resistance components, as detailed in the following. For protein level, seed samples were tested for protein, total amino acids and moisture content using a NIR analyzer calibrated by a wide spectrum of pea seeds compositional analysis using the total Kjeldahl Nitrogen method for protein analysis and total amino acid determination in foodstuffs after acid hydrolysis using an ionic chromatography. Pea legumin and vicilin protein subunits were quantified using SDS PAGE densitometry. The measurement results were summarized into the representative high protein level trait and into the protein composition trait. Evaluation of seed color and cotyledon color was carried out by visual inspection, with the cotyledon color graded as yellow, green or mixture. Powdery mildew field resistance was evaluated by visual inspection after harvest, with plants graded as infected or not infected (resistant).

TPGC Discovery (iii) included genotyping ca. 3200 selected individual plants from 8 populations. The analysis was performed with a panel of 600 markers based on single nucleotide polymorphism (SNP) and directly designed based on the polymorphism found in the parental lines of the populations which were analyzed in depth using GenoPea™ array (Tayeh et al. 2015, Development of two major resources for pea genomics: the GenoPea™ 13.2 K SNP Array and a high-density, high-resolution consensus genetic map, The Plant Journal, Volume 84, Issue 6), Humphry et al. 2011 (Durable broad-spectrum powdery mildew resistance in pea ER1 plants is conferred by natural loss-of-function mutations in PsMLO1, Molecular Plant Pathology 12: 866-878) and independent RNA sequence analysis. The Panel was designed to maximize the chance to have the largest number of common segregate SNP’s in order to create highly similar linkage maps for all observed populations. The computation of linkage maps was executed on each linkage F2 population based on the genotyping results. Linkage maps were computed with MultiPoint™, an interactive package for ordering multilocus genetic maps, and verification of maps based on resampling techniques. Discovery of QTLs that are related to high protein level was carried out with the MultiQTL™ package, based on the linkage maps that were merged by Multipoint and the F2 population phenotype data, and using multiple interval mapping (MIM). MultiQTL™ significance was computed with permutation, bootstrap tools and FDR (false discovery rate) for total analysis. The linkage maps of all eight F2 populations and the information of the high protein level traits over all genotyped plants belonging to those populations were analyzed and used to predict the QTLs in a “one trait to one marker” model, in which for all markers that constructed the linkage maps, each trait was tested independently against each one of the markers. In the provided examples, 54 markers were found to be related to protein content and to protein composition, between 2-19 markers per population. Out of these markers, four markers (linked to corresponding QTLs 1, 3, 4, 5 in Table 7) were selected to use for identifying high protein lines and two markers (linked to QTL 2 and 6 in Table 7) were found to be associated with the protein composition trait (see also Table 8). In addition, one marker (linked to QTL 7 in Table 7) was found to be associated with the semi-leafless trait, two markers (linked to QTL 10 and 11 in Table 7) were found to be associated with the yellow cotyledon trait, and two markers (linked to QTLs 8 and 9 in Table 7) were found to be associated with the powdery mildew resistance trait. In general, the populations presented different markers that related to high protein levels. However, subsets of common markers were found to be shared by multiple populations, and are referred to herein as marker cassettes. The significance and co-occurrences of the high protein level markers were evaluated using an algorithm that related the genotype-phase of each marker to respective QTLs and traits in linkage F2 in each population, for populations in different environments. The occurrence of high protein level markers in two or more linkage F2 population (repetitive markers) strengthened its significance as representative for high protein level QTL. In addition, the co-occurrence of non-repetitive and repetitive markers related to high protein level in a given population was observed for the design of the marker cassettes that provide the genetic signature for high protein level pea lines.

Following TPGC Discovery (iii), an in-silico breeding program (iv) was established to process the TPGC blend (including combinations of QTLs for different plants) to simulate and predict the genotypic states of self, cross-self and hybrid plant with respect to the QTLs and their predicted effects on each phase of the markers for the high protein level trait. The in-silico breeding program was constructed to yield millions of in silico selfing combinations, which were then bred and evaluated up to F8 - to measure the potential for each of the genotyped plants to acquire the high protein level in the right combination at the right phase. The analysis resulted in identifying ca. 200 plants having the highest score for high protein level, which were thus chosen for the actual selfing and cross-selfing procedures. Under this procedure, QTLs from different population were combined to yield plants containing new and unique cassettes of QTLs and yielding high protein levels.

The high protein level pea lines were then validated as retaining the trait in the following generations by genotyping the offspring to verify they maintain the identified marker cassettes. Specifically, the parental lines of linkage F2 populations together with 190 different pea cultivars (landraces and commercial varieties) were genotyped based on high protein level markers of all populations. The cassettes detailed in Table 7 were found to wholly differentiate the developed high protein lines and the rest of the pea cultivars screened.

Disclosed pea lines that reach high protein content larger than 25%, e.g., in various lines, 26%, 27%, 28%, 30%, 35% or intermediate values (as dry weight percentage). Such high protein content allows using the disclosed pea lines for producing high protein concentrate (>65% dry weight percentage) for textured vegetable products (TVP) such as meat replacements. Moreover, advantageously, disclosed pea lines that enable the use of a sustainable and cost-effective protein enrichment process using dry fractionation and/or air classification as a processing method, which do not require large amounts of water and solvents (or even not requiring addition of any water or solvents, and having a significantly lower energy consumption) as the wet fractionation methods applied to prior art pea lines with lower protein content. Enabling dry fractionation to yield higher purity protein concentrate products also opens the possibility to use disclosed pea lines to produce highly nutritious and functional TVPs for human consumption, rather than the more common prior art use of pea concentrate for animal feed (typically 55% protein weight percent in commercial pea concentrates), due to their lower protein content and poor quality. Specifically, different disclosed pea lines were used to produce by dry fractionation texturized pea protein products having 63%, 64%, 66%, 68% and 72% protein. Generally, disclosed pea lines may be used to produce by dry fractionation texturized pea protein products having any of above 60%, above 65% or above 70% protein.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claimed is:

1. A dairy analogue or protein-fortified beverage comprising a pea protein concentrate (PPC), the PPC comprising 65 wt% or more protein (dry base), 2.5 wt% or less starch, 2.8 wt% or more sucrose, and a 1.3% protein fraction of vicilin 1 and 2 or lower.

2. The dairy analogue or protein-fortified beverage of claim 1, wherein the PPC comprises 68 wt% or more protein (dry base).

3. The dairy analogue or protein-fortified beverage of claim 1, wherein the PPC has a gelation firmness of 0.8 N or less, a high solubility of 75%PDI or more and a low water holding capacity (<0.6 gH₂O/gPPC).

4. A meat analogue or texturized pea protein comprising a pea protein concentrate (PPC), the PPC comprising 65 wt% or more protein, 2.2 wt% or less sucrose, and a 1.3% protein fraction of vicilin 1 and 2 or higher.

5. The meat analogue or texturized pea protein of claim 4, wherein the PPC comprises 68 wt% or more protein.

6. The meat analogue or texturized pea protein of claim 4, wherein the PPC has a sodium content of 5 mg/100 g or less.

7. The meat analogue or texturized pea protein of claim 4, wherein the PPC has a gelation firmness of 0.8 N or more, and solubility of 65%PDI or more.

8. The meat analogue or texturized pea protein of claim 4, without any pea protein isolate (PPI) or with up to 20% mixed PPI.

9. The meat analogue or texturized pea protein of claim 4, produced by high-moisture extrusion (HME) or by low-moisture extrusion (LME).

10. Pea protein concentrate (PPC) comprising 65 wt% or more protein (dry base) and 2.2 wt% or less starch.

11. The PPC of claim 10, comprising 68 wt% or more protein (dry base), and optionally a vicilin 6 content above 1% or protein fraction.

12. The PPC of claim 10, comprising between 65% and 75% protein (dry base), less than 2.5 wt% starch, less than 5 mg/100 g sodium, between 0.02% and 2.2% Vicilin 1,2 and between 1.8 and 3.6 g/100 g sucrose.

13. The PPC of claim 10, comprising a 1.3% protein fraction of vicilin 1 and 2 or lower.

14. The PPC of claim 10, having a gelation firmness of 0.8 N or less, 2.3% (% dry base) or more sucrose and configured for use in dairy analogues, protein-fortified beverages, protein fortified bars and baked goods.

15. The PPC of claim 10, comprising a 1.3% protein fraction of vicilin 1 and 2 or higher.

16. The PPC of claim 10, having a gelation firmness of 0.8 N or more, 2.2% (dry base) or less sucrose, and configured for use in at least one of: texturized proteins, meat analogues, baked goods, and pasta.

17. The PPC of claim 16, further configured for use in meat analogues without addition of pea protein isolate (PPI), or with addition of up to 20% PPI.

18. The PPC of claim 10, produced by dry fractionation.

19. The PPC of claim 10, comprising: a plurality of loci associated with a corresponding plurality quantitative trait loci (QTLs) having a corresponding plurality of nucleic acid genetic markers that are associated with a plurality of phenotypic traits of pea plants with seeds having high protein content of at least 28% from which at least part of the PPC is prepared, wherein:
the phenotypic traits comprise a high protein content of the seeds of at least 25% and semi-leafless and powdery mildew resistance,
the plurality of QTLs and corresponding markers comprise at least three QTLs and corresponding markers,
the QTL and marker associated with the semi-leafless trait comprise QTL 7 with corresponding markers set forth in Seq. IDs 13 or 14, and
the pea plants and the seeds are homozygous with respect to Seq. ID 13 or heterozygous at QTL 7.
